(19) United States
(12) Patent Application Publication
(10) Pub. No.: US 2015/0140289 A1
(43) Pub. Date: May 21, 2015

(54) METHOD OF PATTERNING A BIORESORBABLE MATERIAL

(71) Applicant: Bioflex Devices, Dublin (IE)

(72) Inventors: Marc FERRO, Aix en Provence (FR); George MALLIARAS, Fuveau (FR)

(21) Appl. No.: 14/547,774

(22) Filed: Nov. 19, 2014

Related U.S. Application Data
(60) Provisional application No. 61/906,177, filed on Nov. 19, 2013.

(51) Int. Cl. A61F 2/00

(52) U.S. Cl. CPC A61F 2/00; A61F 2210/0004; A61F 2210/0076
USPC 428/195.1; 216/49

(57) ABSTRACT

A method of patterning a bioresorbable material includes providing a fluoropolymer layer in a first pattern over a layer of bioresorbable material. A patterned bioresorbable material is formed by selectively removing the bioresorbable material in areas not covered by the first pattern of fluoropolymer. The fluoropolymer layer may optionally be provided using a photosensitive fluoropolymer along with fluorinated solvents for coating and developing images. The disclosed methods may provide patterned bioresorbable materials having simple or complex shapes, coarse or fine features. In some embodiments, high volume manufacturing methods such as photolithography may be used. Flexible bioelectronic devices may be provided with patterned bioresorbable materials to aid in application into biological targets, but which later bioresorb thereby imparting higher flexibility to the bioelectronic device. In some embodiments, using flexible bioelectronic devices having patterned bioresorbable material corresponding to the device shape can reduces local stresses on biological systems.

### CROSS-REFERENCE TO RELATED APPLICATIONS

The present application claims the benefit of U.S. Provisional Application No. 61/906,177, filed Nov. 19, 2013, which is incorporated herein by reference.

### BACKGROUND

The present disclosure relates to the field of bioresorbable materials, in particular, methods of patterning bioresorbable materials and their use in medical applications.

Bioresorbable materials have many possible medical applications due to their ability to be broken down in the body. For example, the bioresorbable material may be loaded with a pharmaceutically active agent to produce a drug delivery device [see Nicolson et al., US20100317745]. Bioresorbable materials have been used to make scaffolding for tissue and bone regeneration that does not need to be later removed [see Hutmacher et al., JOMI on CD-ROM, 1996 May, 667-678]. Bioresorbable materials have been used as temporary barriers, e.g., as artificial skin in burn treatments. Flexible, implantable bioelectronic devices have been proposed wherein bioresorbable materials are added to increase structural strength during implantation, but are later broken down in the body [see Hetke et al., US20090132042 and Rogers et al., US20110230747].

In some applications, bioresorbable materials are simply provided as an unpatterned coating around a device. In other applications, the bioresorbable material may be mechanically cut or otherwise machined into a desired shape, which can be difficult if the shape includes fine features. In some applications, bioresorbable materials may be shaped by casting into a conventional mold [US20090132042], which can also be difficult if the shape includes fine features. As a variation, nanoimprinted molds have been suggested for patterning biomaterials requiring fine features [see DeSimone et al. Chem. Soc. Rev., 2006, 35, 1095-1104], but nanoimprinting can be difficult on a large scale. Some researchers have imparted photosensitivity (photo-crosslinking groups) to polyethylene glycol to enable direct patterning by light [see Willson et al., Chem. Mater. 2004, 16, 5574-5580 and Pishko et al., Langmuir 2001, 17, 5440-5447]. Such chemical modifications, however, can alter the bioresorbable properties.

### SUMMARY

In accordance with an embodiment of the present disclosure, a method of patterning a bioresorbable material comprises: providing a layer of bioresorbable material; providing a fluoropolymer layer in a first pattern over the layer of bioresorbable material; and selectively removing the bioresorbable material in areas not covered by the first pattern of fluoropolymer, thereby forming a patterned bioresorbable material.

In accordance with another embodiment of the present disclosure, a method of forming a patterned bioelectronic device comprises: providing a flexible substructure having at least one bioelectronic feature, at least a portion of which is electrically conductive or semiconductive; providing a layer of bioresorbable material over the flexible substructure including the bioelectronic feature; providing a layer of a fluoropolymer in a first pattern covering the bioresorbable material thereby forming a second pattern of non-covered bioresorbable material, wherein the total fluorine content of the fluoropolymer is in a weight range of 15 to 60%; and selectively removing the second pattern of non-covered bioresorbable material, thereby forming a layer of patterned bioresorbable material corresponding to the first pattern and a pattern of uncovered substructure corresponding to the second pattern.

In accordance with another embodiment of the present disclosure, a bioelectronic device comprises: a flexible substructure including a bioactive portion having a first side and a second side, wherein the bioactive portion is provided in a first pattern and includes at least one bioelectronic feature, at least a portion of which is conductive or semiconductive, provided as part of the first side; and a layer of bioresorbable material provided over the first side of the bioactive portion in a pattern corresponding to the first pattern, wherein the bioelectronic device includes substantially no bioresorbable material on the second side of the bioactive portion.

In certain embodiments, the disclosed methods can provide patterned bioresorbable materials having simple or complex shapes, coarse or fine features. In some embodiments, high volume manufacturing methods such as photolithography may be used. In some embodiments, flexible bioelectronic devices can be provided with patterned bioresorbable materials that initially provide increased mechanical strength for handling and application to biological targets, but which later bioresorb thereby imparting higher flexibility to the bioelectronic device. In some embodiments, using flexible bioelectronic devices having patterned bioresorbable material corresponding to the device shape can reduces local stresses on biological systems.

### BRIEF DESCRIPTION OF DRAWINGS

FIG. 1 is a flow chart depicting the steps in an embodiment of the present disclosure;

FIG. 2 is a cross-sectional view depicting a layer of bioresorbable material according to an embodiment of the present disclosure;

FIG. 3A-3C is a series of cross-sectional views depicting the various stages in the formation of a patterned fluoropolymer over a bioresorbable material according to an embodiment of the present disclosure;

FIG. 4A-4D is a series of cross-sectional views depicting the various stages in the formation of a patterned fluoropolymer over a bioresorbable material according to another embodiment of the present disclosure;

FIG. 5 is a cross-sectional view depicting a patterned bioresorbable material according to an embodiment of the present disclosure;

FIG. 6 is a flow chart depicting the steps in another embodiment of the present disclosure;

FIG. 7 is a cross-sectional view of a flexible substructure according to an embodiment of the present disclosure;

FIG. 8A-8D is a series of cross-sectional views depicting the various stages in the formation of the flexible substructure of FIG. 7 according to an embodiment of the present disclosure;

FIG. 9 is a cross-sectional view depicting a layer of bioresorbable material provided over the flexible substructure from FIG. 7 according to an embodiment of the present disclosure;

FIG. 10 is a cross-sectional view depicting a patterned fluoropolymer provided over the bioresorbable material from FIG. 9 according to an embodiment of the present disclosure;

FIG. 11 is a cross-sectional view depicting a patterned bioresorbable material formed from the structure of FIG. 10 according to an embodiment of the present disclosure;

FIG. 12 is a cross-sectional view depicting a patterned bioelectronic device and patterned flexible substructure formed from the structure of FIG. 11 according to an embodiment of the present disclosure;

FIG. 13 is a cross-sectional view of the patterned bioelectronic device from FIG. 12 with the patterned fluoropolymer removed according to an embodiment of the present disclosure;

FIG. 14 is a cross-sectional view of the patterned bioelectronic device from FIG. 13 removed from the carrier substrate according to an embodiment of the present disclosure;

FIG. 15A is a plan view depicting a first intermediate structure in the formation of an array of patterned bioelectronic devices according to an embodiment of the present disclosure;

FIG. 15B is a magnified plan view of FIG. 15A in the region highlighted by circle A.

FIG. 15C is a plan view depicting a second intermediate structure in the formation of an array of patterned bioelectronic devices according to an embodiment of the present disclosure;

FIG. 15D is a magnified plan view of FIG. 15C in the region highlighted by circle B;

FIG. 15E is a cross-sectional view taken along cut line C-C drawn in FIG. 15D;

FIG. 16 is a plan view depicting a third intermediate structure in the formation of an array of patterned bioelectronic devices according to an embodiment of the present disclosure;

FIG. 17A is a plan view depicting an array of patterned bioelectronic devices according to an embodiment of the present disclosure;

FIG. 17B is a magnified plan view of FIG. 17A in the region highlighted by circle D;

FIG. 17C is a cross-sectional view taken along cut line E-E drawn in FIG. 17B after removal from the carrier substrate;

FIG. 18 is a cross-sectional view of a protected flexible substructure according to an embodiment of the present disclosure;

FIG. 19 is a cross-sectional view of the structure from FIG. 18 now with a layer of bioresorbable material and a patterned fluoropolymer over the bioresorbable material according to an embodiment of the present disclosure;

FIG. 20 is a cross-sectional view of a patterned bioelectronic device having a patterned protected flexible substructure according to an embodiment of the present disclosure;

FIG. 21 is a cross-sectional view of a patterned bioelectronic device removed from the carrier substrate according to an embodiment of the present disclosure;

FIG. 22 is a flow chart depicting the steps in another embodiment of the present disclosure;

FIG. 23 is a cross-sectional view depicting a layer of bioresorbable material according to an embodiment of the present disclosure;

FIG. 24A-24E is a series of cross-sectional views depicting the formation of a protected flexible substructure over the bioresorbable material of FIG. 23 according to an embodiment of the present disclosure

FIG. 25 is a cross-sectional view of a patterned bioelectronic device having a patterned protected flexible substructure according to an embodiment of the present disclosure; and

FIG. 26 is a cross-sectional view of a patterned bioelectronic device removed from the carrier substrate according to an embodiment of the present disclosure;

### DETAILED DESCRIPTION

It is to be understood that the attached drawings are for purposes of illustrating the concepts of the disclosure and may not be to scale.

A “bioresorbable material” is one that is susceptible to being chemically broken down into lower molecular weight moieties by reagents that are naturally present in a biological environment. In an in-vivo application, for example, the chemical moieties may be assimilated into human or animal tissue. The term is used broadly and is meant to include any bio-mechanism that solubilizes or breaks down the bioresorbable material. For example, the bio-mechanism may be by a biodegradation whereby a biological agent such as an enzyme, a microbe or cell is responsible for the degradation. The bio-mechanism may be by bioerosion whereby physical processes (such as dissolution) or chemical processes (such as chemical bond cleavage) or both act on the material to make it more water soluble. The bio-mechanism may be by cellular activity such as phagocytosis.

The desired rate of bioresorption will depend upon the application, and suitable material sets can be selected to meet the particular needs. With tissue scaffolds, barriers and sutures, the bioresorption rate may, for example, be chosen to approximately match the expected tissue healing rate. With drug or diagnostic imaging material delivery, the bioresorption rate may, for example, be chosen to achieve the desired dosage rate. For bioelectronic devices, the bioresorption rate may, for example, be chosen to approximately match the expected life of the device. Alternatively, it may be chosen so that the bioresorbable material is removed from the bioelectronic device very quickly, e.g., to quickly impart increased flexibility or to avoid interference of the bioresorbable material with an active electronic site. Bioresorbable materials of the present disclosure are generally at least 50% completely resorbed within 2 years. In an embodiment, bioresorption is at least 50% complete within 1 year, or in another embodiment within 1 month, or in another embodiment within 1 week, or in another embodiment within 1 day, or in another embodiment within 1 hour, or in another embodiment within 1 minute.

There are many known bioresorbable materials that may be useful in various embodiments of the present disclosure, some of which are disclosed in US20110230747, US20090132042, and US20100317745, the entire contents of which are incorporated by reference. Some non-limiting examples of useful bioresorbable materials include polymeric materials such as polyethylene glycol (PEG), polylactic acid (PLA), polyglycolic acid (PGA), polyvinylalcohol (PVA), polyacrylic acid, (PAA) polycaprolactone (PCL), collagen, polyphosphazenes, polyester-ethers, polyamino acids, silks, their derivatives and combinations (e.g., mixtures or copolymers) thereof. In an embodiment, the bioresorbable material may include a bioresorbable organic polymer having some solubility (e.g., at least 0.001% w/w) or some swellability (1% v/v swell or larger) in water.

In some embodiments, the bioresorbable material may incorporate a pharmaceutical or diagnostic imaging material that is released over time as the bioresorbable material itself resorbs. There is no particular limitation to the types of drugs or imaging materials or their intended function. They may, for example, be small molecules, proteins, nucleic acids, polymers or nanoparticles. The materials may function as osteoinductive agents, antibiotics, anesthetics, growth factors, cells, anti-tumor agents, anti-inflammatory agents, antiparasitics, antigens, adjuvants, cytokines, hormones, diagnostic tags or the like.

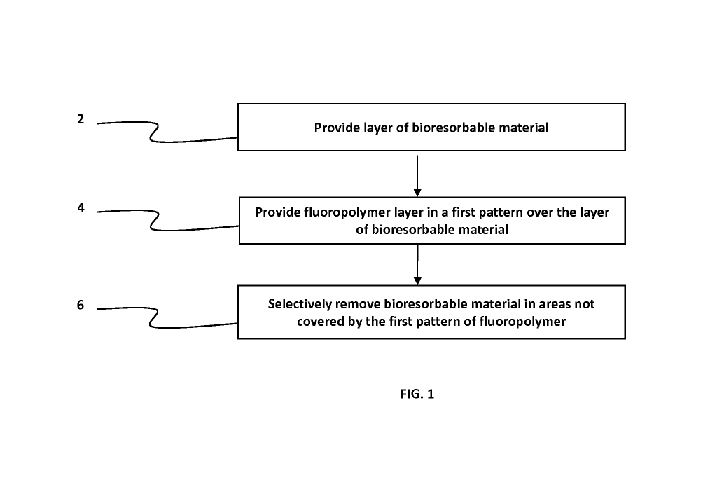

A flow diagram for an embodiment of the present disclosure is shown in FIG. 1, and includes the step 2 of providing a layer of bioresorbable material, the step 4 of providing a fluoropolymer layer in a first pattern over the bioresorbable material, and the step 6 of selectively removing bioresorbable material in areas not covered by the first pattern of fluoropolymer. Each step is discussed in more detail, below.

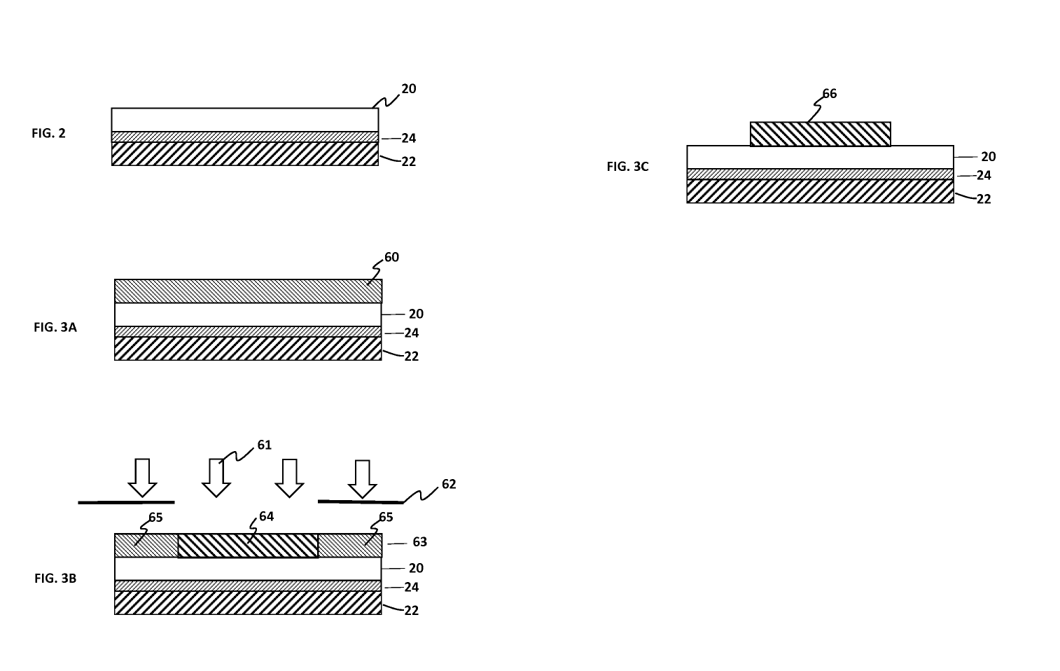

Turning to an embodiment shown in FIG. 2, a layer of bioresorbable material 20 is provided over an optional carrier substrate 22 along with an optional intervening release layer 24. As mentioned above, the choice of bioresorbable material will depend largely upon the particular application.

The layer of bioresorbable material 20 may be provided as a preformed sheet or coated from a melt or solution, e.g., by spin coating, curtain coating, doctor-blade coating, dip coating, ink jet coating, spray application or the like. There is no particular limitation on the thickness of the layer of bioresorbable material other than it cannot be so thick that it will not properly pattern (see below) and that the final thickness will serve its intended purpose. The layer of bioresorbable material 20 may be substantially flat, or alternatively, may be substantially non-flat (e.g., having a structured or rounded surface) as long as a surface of the bioresorbable material is capable of functionally receiving the first pattern of fluoropolymer. In an embodiment, the layer of bioresorbable material may have a thickness in a range of 0.5 to 1000 μm, or in another embodiment, a range of 10 to 100 μm, or in another embodiment, a range of 20 to 60 μm, or in another embodiment in a range of 0.5 to 10 μm.

The optional carrier substrate 22 is particularly useful when coating the bioresorbable material from a solution. Such substrates are preferably flat and may be formed, e.g., from sheets or wafers of glass, silicon, metal, ceramic, plastic or combinations thereof. The optional release layer 24 can be useful when one desires to separate a structure having a patterned base layer (see below) from a carrier substrate. The release layer may be a thin layer that simply lowers adhesion between layers (e.g., a layer of surfactant) or it may be a layer that has reasonable adhesion at first, but can be activated in some way, e.g., by thermal or light activation, to promote release when desired. Such materials are known in the art and some are used, e.g., as “light-to-heat conversion” layers or “transfer assist” layers in thermal and laser transfer from donor sheets to receivers. Some non-limiting examples of light-to-heat conversion layers can be found in WO 2008/010982, which is incorporated by reference herein. In an embodiment, the optional release layer 24 is a fluorinated polymer release layer soluble in a fluorinated release agent, e.g., a hydrofluoroether.

A fluoropolymer layer having a first pattern is provided over the layer of bioresorbable material. Many methods of applying or forming the patterned fluoropolymer are available. Some non-limiting examples include ink jet depositing a liquid containing a fluoropolymer, patterned thermal transfer of a dry fluoropolymer from a donor sheet, and flexographic printing of a liquid containing a fluoropolymer. In a preferred embodiment, the layer of patterned fluoropolymer is provided by coating a solution comprising a fluorinated solvent and a fluoropolymer and applying photolithographic methods, e.g., as disclosed in U.S. patent application publications 2011/0159252 and 2010/0289019, the entire contents of which are incorporated by reference. A generally flat layer of bioresorbable material simplifies coating and photolithography. In an embodiment, an intervening layer of another material may be provided between the patterned layer of fluoropolymer and the layer of bioresorbable material. Such other optional material layer and method of application should be selected so that it is compatible with the bioresorbable material (e.g., in an embodiment, parylene or other polymers based on p-xylene derivatives may be used as an intervening layer). In a preferred embodiment, the fluoropolymer is provided in direct contact with the layer of bioresorbable material from a solution comprising a fluorinated solvent. It has been found that fluorinated solvent-based solutions have been generally found to be “orthogonal” in solubility relative to bioresorbable materials. Thus, application of a patterned fluoropolymer using fluorinated solvents is particularly versatile.

FIGS. 3A-3C illustrate an embodiment for forming a fluoropolymer layer in a first pattern using a photosensitive fluoropolymer. In FIG. 3A, a layer of photosensitive fluoropolymer 60 is formed by applying over the layer of bioresorbable material a composition comprising a photosensitive fluoropolymer material provided in a first fluorinated solvent (a coating solvent). The layer of photosensitive fluoropolymer 60 may include any suitable fluorinated material that can be selectively exposed to appropriate radiation to form exposed and unexposed areas having differential solubility in a fluoropolymer developing agent. The photosensitive fluoropolymer can be negative tone or positive tone, and as described below, there are numerous options for its chemical composition and image development. In the illustrated embodiment, the photosensitive fluoropolymer is negative tone and developable in solution comprising one or more fluorinated solvents. For example, the fluorinated solvent may be a perfluorinated compound, a hydrofluorocarbon, or a hydrofluoroether.

Referring now to FIG. 3B, a photomask 62 is provided between radiation source emitting radiation 61 (e.g., “i-line” UV light at 365 nm) and the layer of photosensitive fluoropolymer (e.g., that is sensitive to 365 nm radiation), thereby forming an exposed layer of photosensitive fluoropolymer 63 having a pattern 64 of exposed photosensitive fluoropolymer and a pattern 65 of unexposed photosensitive fluoropolymer. In FIG. 3C, the exposed layer of photosensitive fluoropolymer 63 is then contacted with a photosensitive fluoropolymer developing agent, preferably having at least 50% by volume of a second fluorinated solvent (that may be the same as or different from the first fluorinated solvent), to selectively remove unexposed areas of the photosensitive fluoropolymer thereby forming a first pattern of fluoropolymer 66. Contacting can be accomplished by immersion into the developing agent or by coating the structure with the developing agent in some way, e.g., by spin coating or spray coating. The contacting can be performed multiple times if necessary.

Although drawn as vertical, the sidewalls of the fluoropolymer layer in first pattern 66 may have some other shape after development. Rather than rectangular, its cross section could resemble a trapezoid, an inverted trapezoid (undercut), or some other shape, e.g., one having curved sidewalls.

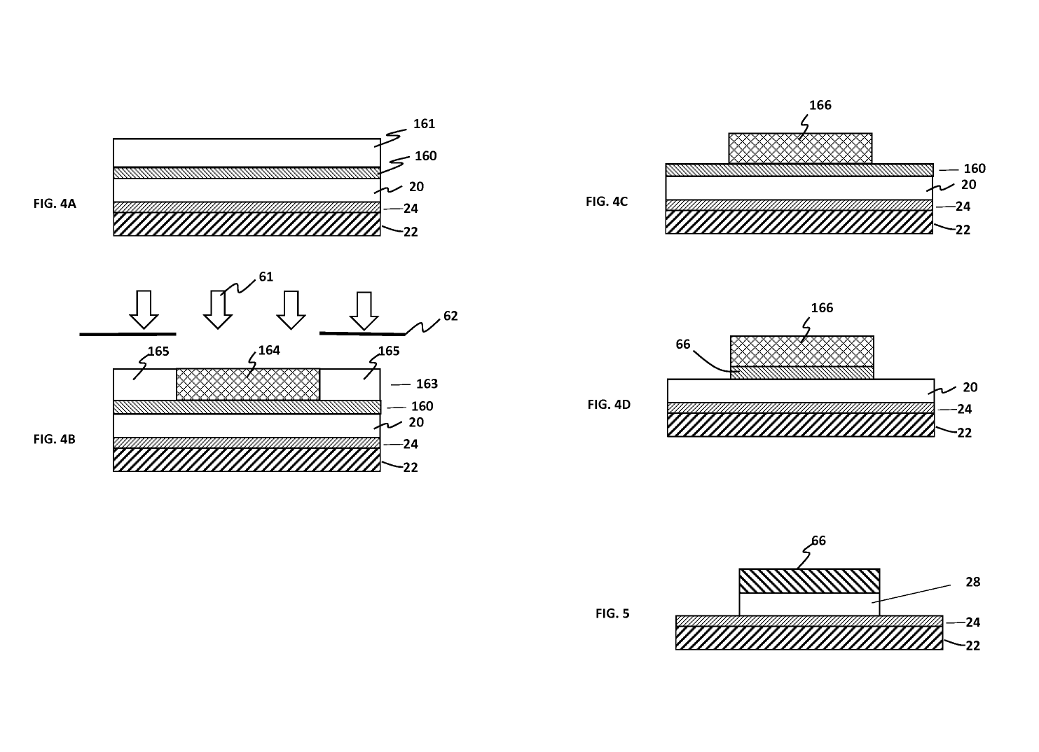

In an alternative embodiment shown in FIGS. 4A-4D, photolithographic patterning is applied to a bilayer structure. In an embodiment shown in FIG. 4A, a layer of an initially non-patterned fluoropolymer 160 is applied over the layer of bioresorbable material, e.g., by coating from a solution or by dry film transfer from a donor sheet. In this embodiment, the fluoropolymer is soluble in one or more fluorinated solvents that do not interact significantly with the layer of bioresorbable material. Next, a layer of a photosensitive second polymer 161 (e.g., a photoresist) is provided over the non-patterned fluoropolymer 160 to form an unpatterned bilayer structure. The photosensitive second polymer 161 may, for example, be coated from an organic or aqueous solution in which the underlying non-patterned fluoropolymer is not soluble. The photosensitive second polymer 161 may be any conventional photoresist or photopolymer that can be coated and developed using aqueous or organic solvents that do not deleteriously interact with the underlying fluoropolymer layer. The developed photosensitive second polymer structure should also have low solubility in fluorinated solvents used to pattern the underlying fluoropolymer layer (see below). In an embodiment, the photosensitive second polymer has a total fluorine content by weight of less than 30%, preferably less than 15%. In an embodiment, the photosensitive second polymer has a total fluorine content of less that 1% by weight.

Referring now to FIG. 4B, a photomask 62 is provided between radiation source emitting radiation 61 (e.g., “i-line” UV light at 365 nm) and the layer of photosensitive second polymer (e.g., that is sensitive to 365 nm radiation), thereby forming an exposed layer of photosensitive second polymer 163 having a pattern 164 of exposed photosensitive second polymer and a pattern 165 of unexposed photosensitive second polymer. In FIG. 4C, the exposed layer of photosensitive second polymer 163 is then contacted with a second polymer developing solution to selectively remove unexposed areas of the photosensitive second polymer thereby forming a partially patterned bilayer structure including a patterned layer 166 of second polymer over non-patterned fluoropolymer 160. The non-patterned fluoropolymer 160 is not highly soluble in the second polymer developing solution and is not removed at this point (in this embodiment). In an alternative embodiment, the patterned layer 166 of second polymer may be formed by printing.

The partially patterned bilayer structure is contacted with a fluorinated solvent in which the fluoropolymer has some solubility, but not the developed second polymer. As shown in FIG. 4D, this results in selective removal of the fluoropolymer in areas not covered by the second polymer, thereby forming a fluoropolymer layer having a first pattern 66. It should be noted that the solubility of the fluoropolymer in the fluorinated solvent may lead to some harmless undercutting (not shown), but this can be controlled through selection of time, temperature, choice of fluorinated solvents, agitation and the like. In some embodiments, the undercutting is desirable. If the contacting with the fluorinated solvent is done under conditions too aggressive, this may result in lift-off of the second polymer. This is not desired at this point, but may be desirable later on if patterning additional material layers.

Next, bioresorbable material in areas not covered by the first pattern of fluoropolymer are selectively removed, thereby forming a patterned bioresorbable material. For example, the structure from FIG. 3C may be contacted with an etching environment, e.g., a plasma etch or a solvent etch, wherein the first pattern of fluoropolymer acts as an etch barrier to form patterned bioresorbable material 28 as shown in FIG. 5. In the embodiment shown in FIG. 5, release layer 24 was not removed, but in alternative embodiment, the release layer 24 may instead be etched in areas not covered by the first pattern of fluoropolymer. The first pattern of fluoropolymer 66 may optionally be removed or stripped (not shown), e.g., by contacting the structure of FIG. 5 with a stripping agent capable of dissolving the first pattern of fluoropolymer 66. In an embodiment, the stripping agent includes at least 50% by volume of a fluorinated solvent. The patterned bioresorbable material may optionally be removed from the carrier substrate 22 (not shown). Non-limiting methods of such removal include physical peeling or pulling of the patterned bioresorbable material off of the carrier substrate, heating or cooling to cause differential expansion/contraction between patterned bioresorbable material and carrier substrate thereby causing separation, dissolving the release layer 24, or activating the release layer by light or heat to cause separation. In an embodiment, the release layer comprises a fluorinated material such as a fluoropolymer and the stripping agent also dissolves the release layer thereby causing release. In some embodiments, once the bioresorbable material has been patterned, it can be advantageous if subsequent processing steps do not cause the patterned bioresorbable material to exceed its Tg.

The shape of the bioresorbable material depends upon the application. The present method is capable of patterning nearly any desired two-dimensional at high resolution (microns). The patterned bioresorbable material may include features that are round, square, elongated, pointed, serrated, barbed, or the like. The patterned bioresorbable material may be selected so that it has sufficient mechanical strength to allow insertion into a target biological environment, e.g., living tissue. The size and shape can be selected for preparing bone or tissue scaffolding that is tailored to a particular patient. The patterned bioresorbable material may be associated with a bioelectronic device, e.g., a bioelectronic device may be attached to or embedded in the patterned bioresorbable material and the bioelectronic device may have a pattern corresponding to the pattern of the bioresorbable material.

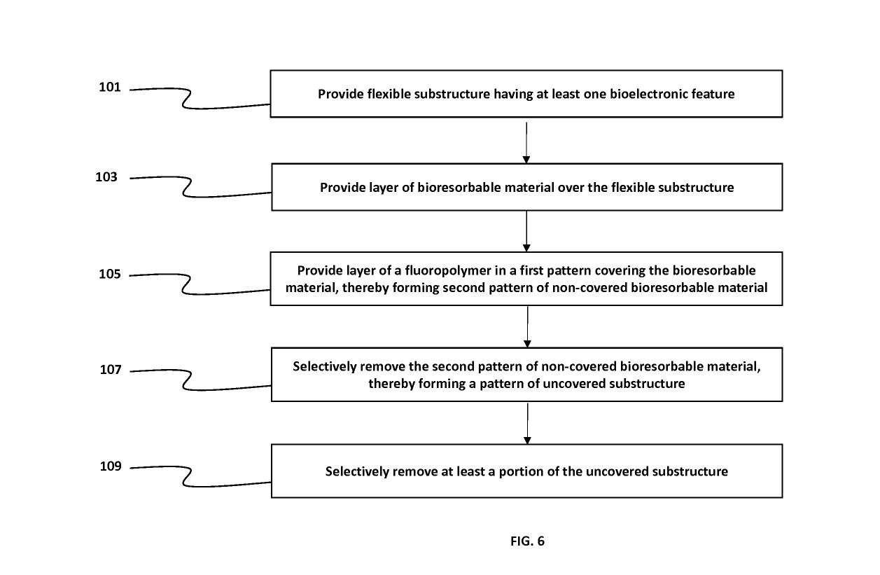

In an embodiment, patterned bioresorbable material is provided as part of a patterned device structure, e.g., a patterned bioelectronic device. A flow diagram of an embodiment is shown in FIG. 6. The method includes the step 101 of providing a flexible substructure having at least one bioelectronic feature, the step 103 of providing a layer of bioresorbable material over the flexible substructure, the step 105 of providing a layer of fluoropolymer in a first pattern covering the bioresorbable material, thereby forming a second pattern of non-covered bioresorbable material, the step 107 of selectively removing the second pattern of non-covered bioresorbable material, thereby forming a pattern of uncovered substrate and a patterned layer of bioresorbable material corresponding to the first pattern. Optionally, the method includes step 109 of selectively removing at least a portion of the uncovered substrate, e.g., by using the first pattern of fluoropolymer and the layer of patterned bioresorbable material as an etch mask. Each step is discussed in more detail, below.

There are many possible configurations that a flexible substructure can take and will depend upon the desired function. A flexible substructure includes at least one bioelectronic feature, at least a portion of which is electrically conductive or semiconductive. The bioelectronic feature may comprise materials such as metals, graphene, carbon nanotubes, nanowires, organic conductors (e.g., conductive polymers such as PEDOT:PSS) organic semiconductors (e.g., those useful in making OTFTs or OLEDs), metal oxides, or nanoparticles. The bioelectronic feature may comprise a single layer or it may include a multilayer structure. A bioelectronic feature is capable of interacting in some way with a biological environment such as living tissue. It may act as a biosensor (e.g., to measure temperature, pressure, pH, chemical concentrations, electrical impulses or the like), a bio-stimulator (e.g., to provide electrical, acoustic or optical stimulation), an ion pump or a drug delivery device. In an embodiment, the bioelectronic feature is an organic electrochemical transistor. The bioelectronic feature(s) is provided in the bioactive portion of the flexible substructure, i.e., the general area of the substructure that is intended to interact with the biological environment. The flexible substructure may optionally have other regions, e.g., an electrical connector portion. These other regions do not directly interact with the biological environment, but serve another purpose, e.g., as to provide electrical connectivity to the bioelectronic feature(s). In an embodiment, the bioresorbable material is patterned so that it is present only in the bioactive portion of the flexible substructure. Although at least a portion of the bioelectronic feature is conductive or semiconductive, this portion may or may not be in direct contact with the biological environment. In some instances the bioelectronic feature may include one or more intervening layers between the conductive or semiconductive portion and the biological environment. Any bioelectronic device formed over the flexible support is considered to be provided on, or as part of, the first side of the flexible substructure.

Turning to FIG. 7, a cross-sectional view is shown for an embodiment of a flexible substructure 112 that comprises flexible support 110, metal electrode 123, electrical insulator 130, and conductive polymer 140. Metal electrode 123 and conductive polymer 140 may, for example, be used as a sensing electrode in a biosensor device. In this embodiment, the metal electrode 123, the conductive polymer 140, and the combination of the two can each be considered bioelectronic feature. Flexible substructure 112 is provided over optional carrier substrate 22 and optional release layer 24, which were described previously. Although the flexible substructure might not be readily flexible while provided on the carrier substrate, it is flexible in a released state. The degree of flexibility required will depend upon the particular application for the substructure. Further, the local flexibility of a substructure may vary along its length or width due to the presence or absence of various features. For example, flexibility at a portion including the metal electrode 123, patterned electrical insulator 130 and conductive polymer 140, may be less than a portion that having only flexible support 110. The term “flexible substructure” in this context means that the substructure is capable bending to an average radius of at 10 m or less, without compromising the intended function of the bioelectronic feature. In an embodiment, the flexible substructure can be bent to an average radius of 1 m or less, or in another embodiment to 100 cm or less, or in another embodiment to 10 cm or less, or in another embodiment to 1 cm or less.

Most or all materials used in a bioelectronic device, e.g., flexible support 110 and patterned electrical insulator 130 are preferably biocompatible. “Biocompatible” refers to a material that does not elicit a serious immunological rejection or significant detrimental effect when it is disposed within an in vivo biological environment. For example, a biological marker indicative of an immune response changes less than 50% from a baseline value when the biocompatible material is contacted or implanted into such biological environment.

Some non-limiting examples of materials that may be used as flexible support 110 and patterned electrical insulator 130 include polyp-xylene) derivatives (parylenes), fluoropolymers, acetal polymers, cellulosic polymers, nylons, polyacrylonitrile polymers, polyamide-imide polymers, polyimides, polyarylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, polyurethanes, styrenic resins, sulfone-based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, polyolefin, flexible glass or any combinations of these. In embodiments where the bioelectronic device is intended for a period of use and later removal, the selected materials (other than the bioresorbable material) should not be readily biodegradable on the timescale of the anticipated device removal.

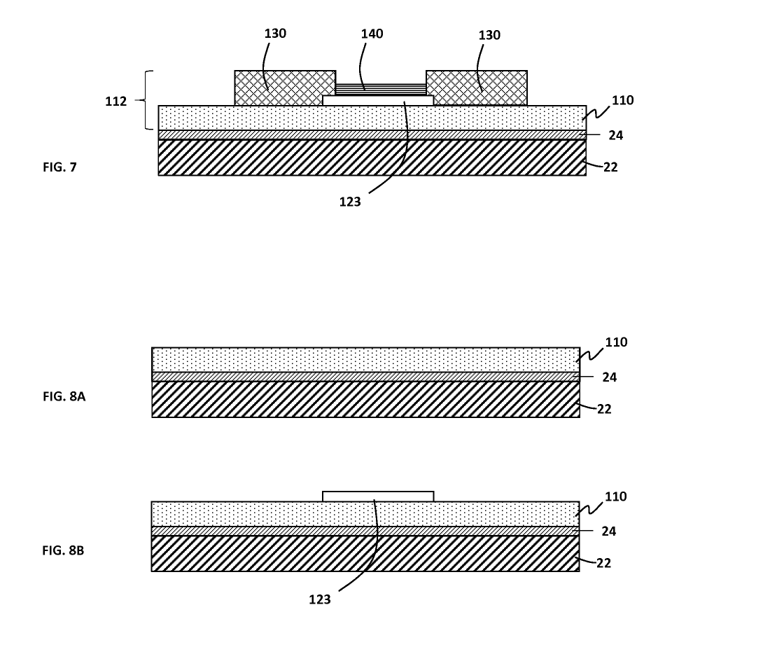

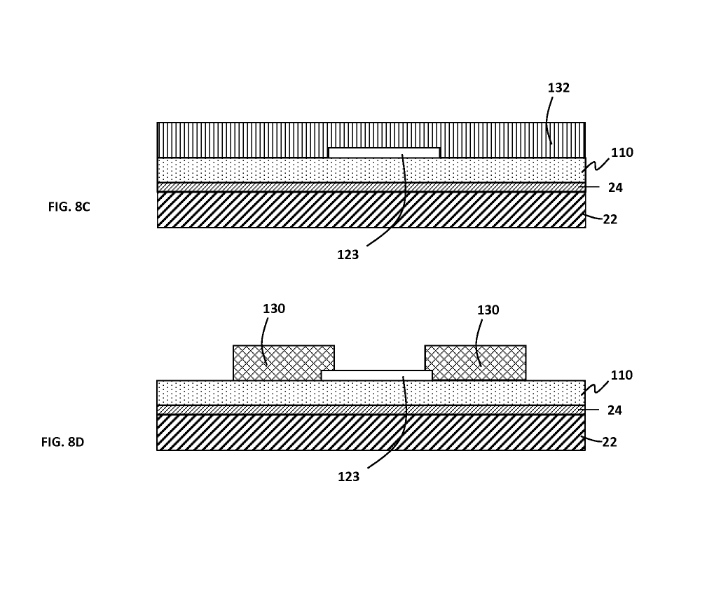

FIG. 8 illustrates a non-limiting embodiment for producing a flexible substructure. In FIG. 8A, a flexible support 110 (e.g., a parylene or a fluoropolymer) is provided over a carrier substrate 22 (e.g., a glass or silicon wafer) and release layer 24 (e.g., a surfactant). Next, as shown in FIG. 8B, an electrically conductive metal electrode 123 (e.g., Au with a Ti adhesion layer) is pattern deposited over the flexible support (e.g., by vapor deposition through a shadow mask or by photolithography). In FIG. 8C, a photopolymer 132 (e.g., a negative tone, cross-linking photosensitive fluoropolymer) is provided over the structure and imaged by photolithography (e.g., exposed through a photomask and developed using a fluorinated solvent) to form patterned electrical insulator 130 that covers the edges of the metal electrode as shown in FIG. 8D. Next a conductive polymer 140 (e.g. PEDOT:PSS) is provided over the metal electrode (e.g., by ink jet deposition, photolithographic patterning, or electropolymerization), thereby forming the flexible substructure 112 shown in FIG. 7. If the conductive polymer 140 is provided by photolithographic patterning, it optionally may be patterned prior to formation of patterned electrical insulator 130, and may optionally have a structure wherein the insulator extends over a top edge portion of the conductive polymer.

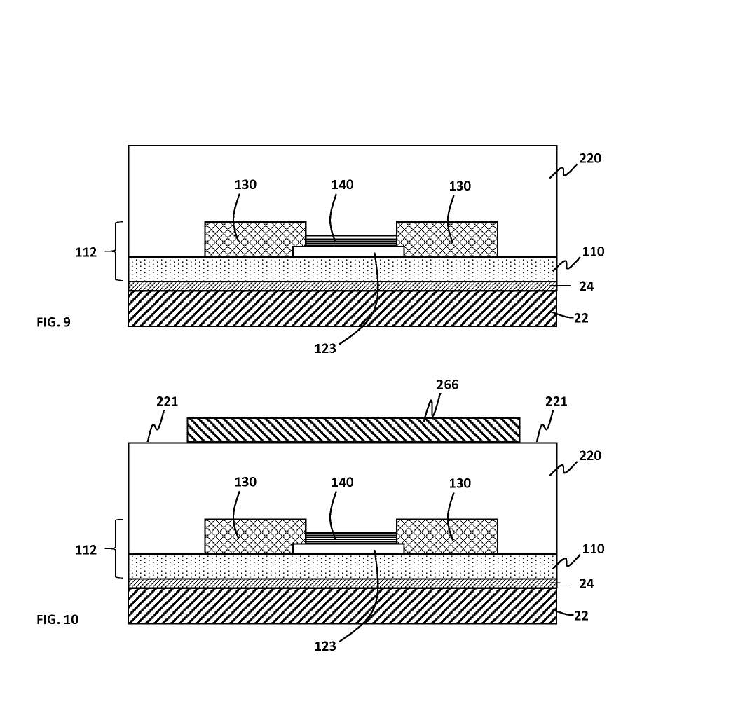

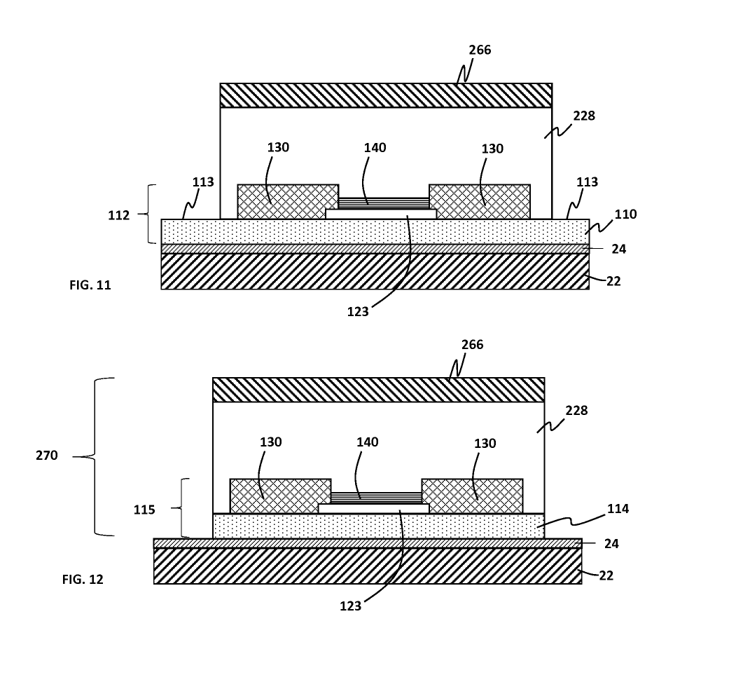

In FIG. 9, a layer of bioresorbable material 220 is provided over the flexible substructure, e.g., by coating. Bioresorbable materials and methods for coating have been discussed above and may be used in the present embodiment, too. In FIG. 10, a layer of a fluoropolymer 266 is provided in a first pattern covering the bioresorbable material thereby forming a complementary second pattern of non-covered bioresorbable material, 221. In FIG. 11, bioresorbable material is selectively removed in areas not covered by the first pattern, i.e., the second pattern of non-covered bioresorbable material is removed. This forms a patterned layer of bioresorbable material 228 corresponding to the first pattern and a pattern of uncovered substructure 113 corresponding to the second pattern. Selective removal of the bioresorbable material can be done, e.g., by an etch step such as a plasma or solvent etch, as previously discussed. The first pattern of fluoropolymer 266 acts as an etch barrier. In an embodiment, at a least a portion of the uncovered substructure is then selectively removed, thereby forming a patterned substructure 115 corresponding to the first pattern and patterned bioelectronic device 270, as shown in FIG. 12. In this embodiment, the uncovered portion of flexible support 110 was removed to form patterned flexible support 114, which forms part of the patterned substructure 115. This removal can also be done by contact with an etching environment, e.g., a plasma etch or a solvent etch. The first pattern of fluoropolymer 266 or the patterned layer of bioresorbable material 228 or the combination acts as an etch barrier for the selective removal of the uncovered substructure. In an embodiment, the selective removal the uncovered substrate may occur as a continuation of the selective removal of the bioresorbable material step. In the embodiment shown in FIG. 12, release layer 24 was not removed, but in alternative embodiment, the release layer 24 may instead be etched in areas not covered by the first pattern of fluoropolymer.

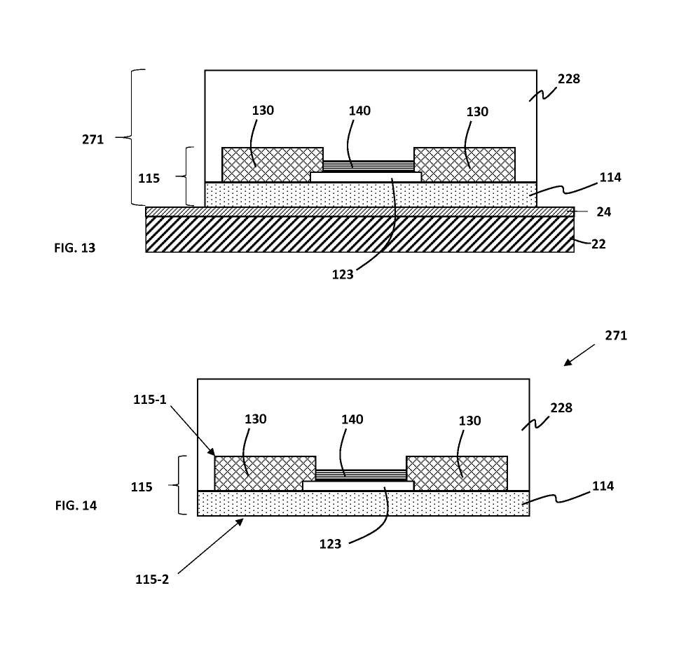

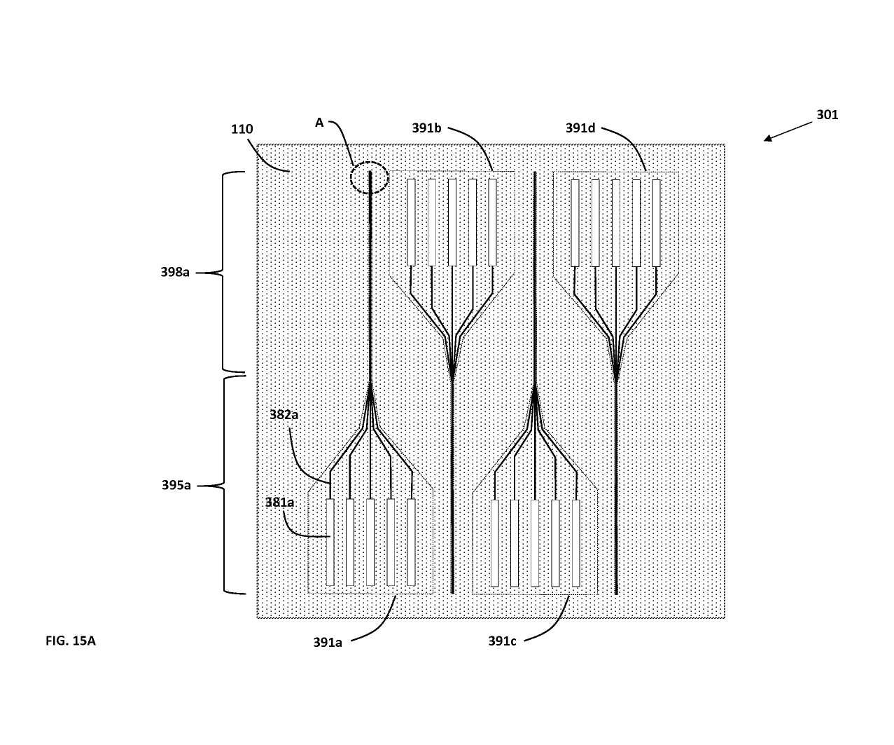

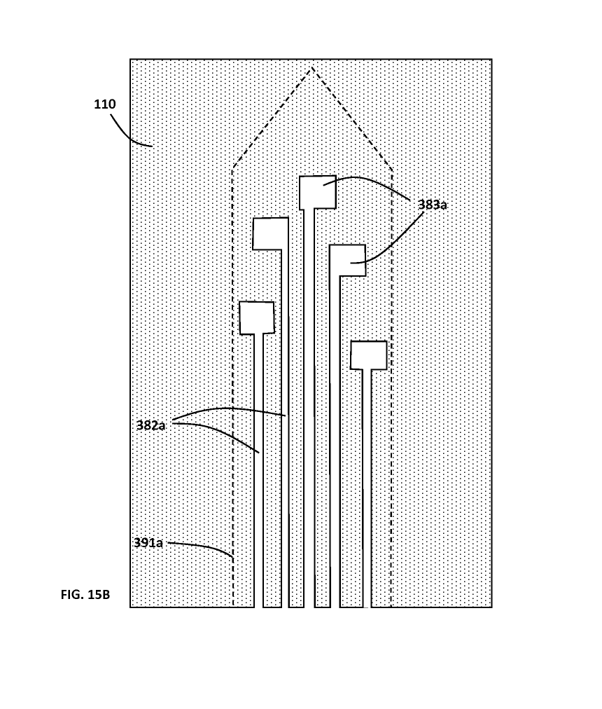

As shown in FIG. 13, the first pattern of fluoropolymer 266 may optionally be removed at some point after the selective removal of bioresorbable material, e.g., by contact with a stripping solution comprising a fluorinated solvent, to form patterned bioelectronic device 271. Patterned bioelectronic device 271 may optionally be removed from the carrier substrate to form a free-standing structure as shown in FIG. 14. Non-limiting methods of such removal include physical peeling or pulling of the patterned bioresorbable material off of the carrier substrate, heating or cooling to cause differential expansion/contraction between patterned bioelectronic device and carrier substrate thereby causing separation, dissolving the release layer 24, or activating the release layer by light or heat to cause separation. In an embodiment, the release layer is has a higher water solubility than the patterned layer of bioresorbable material 228 and water is used to dissolve the release layer. In an alternative embodiment, the release layer is soluble in a fluorinated solvent. In a related embodiment, the release layer is soluble in the same fluorinated solvent used to remove the first pattern of fluoropolymer, thereby allowing device release and fluoropolymer removal to be accomplished in the same step.

Referring again to FIG. 14, patterned bioelectronic device 271 includes patterned flexible substructure 115 having a first side (115-1) and a second side (115-2). The bioelectronic feature forms a portion of the first side. The patterned bioresorbable material 228 is provided only on the first side and there is substantially no bioresorbable material on the second side. By “substantially no bioresorbable material”, it is meant that if any bioresorbable material is present on the second side, its total mass is less than 10% of the mass of patterned bioresorbable material 228 on the first side. In this embodiment, the bioresorbable material is provided in direct contact with the bioelectronic feature.

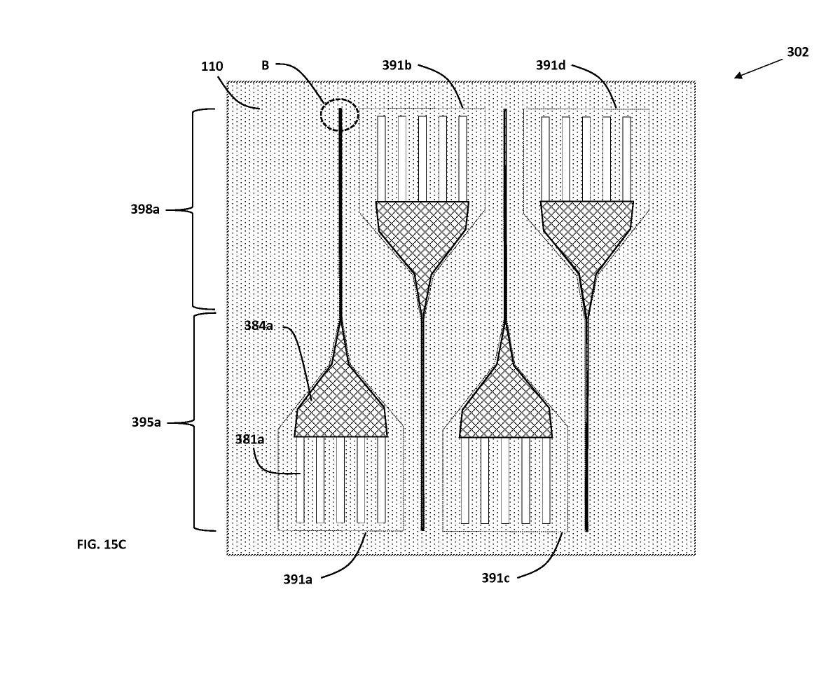

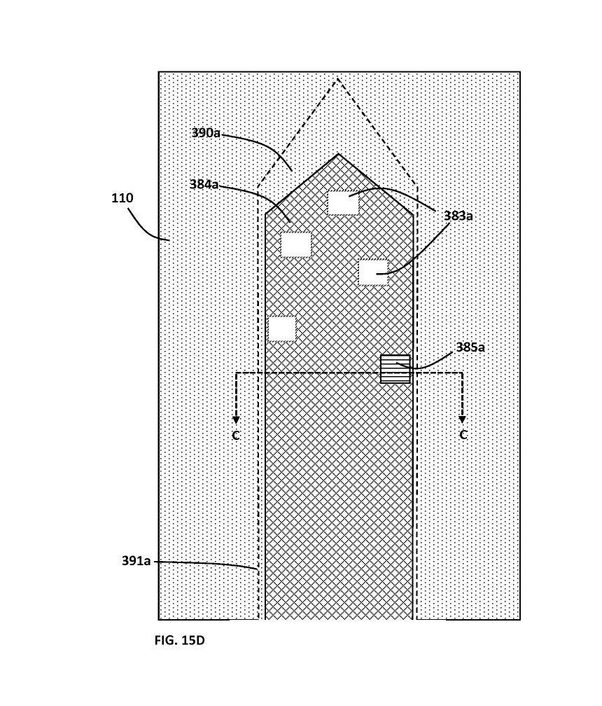

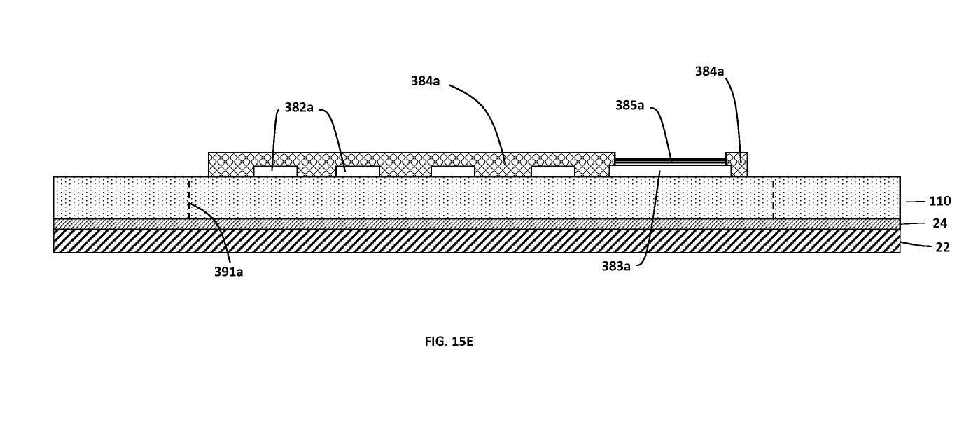

FIGS. 15A-15E illustrate steps for forming an array of four bioelectronic devices, 391a, 391b, 391c, and 391d, according to an embodiment of the present disclosure. FIG. 15A shows a plan view of a first intermediate structure 301 having flexible support 110 provided over a carrier substrate 22 (not visible in this view) and release layer 24 (not visible in this view). Although not yet fully formed, the predetermined shape of each bioelectronic device, 391a through 391d, is outlined by dashed lines. Each bioelectronic device has an electrical contact portion 395 (illustrated as 395a for bioelectronic device 391a) and a shank portion 398 (illustrated as 398a for bioelectronic device 391a). The electrical contact portion 395 include electrical contact pads 381 (illustrated as 381a for bioelectronic device 391a), that provide connection to electronic driving circuitry (not shown). Conductive traces 382 (illustrated as 382a for bioelectronic device 391a) provide electrical connection (electrical communication) from the electrical contact pads 381 to electrode pads near the far tip of the shank. The electrode pads are small and are not shown in FIG. 15A.

The tip of shank portion 398 corresponding to bioelectronic device 391a of the first intermediate structure 301 is highlighted by circle A in FIG. 15A and a magnified plan view is shown in FIG. 15B. Five electrode pads 383a (bioelectronic features) are provided near the pointed tip of the shank and each are individually electrically connected to conductive traces 382a that lead back to electrical contact pads 381a shown in FIG. 15A. In the present embodiment, the shank may be considered a bioactive portion of the flexible substructure, as it includes several bioelectronic features. The shank has an elongated shape and the bioelectronic features are provided at a distal end relative to the electrical contact portion. In other embodiments, the shank may include additional physical features to aid in penetrating tissue (e.g., serrated edges) or holding onto tissue (e.g., barbs). These are easily patterned by using methods of the present disclosure.

Patterned contact pads 381, conductive traces 382 and electrode pads 383 can optionally be formed in a common step and are electrically conductive in the present embodiment. There is no particular limitation on choice of conductive material for these structures. For example, the conductive material may be a metal, a metal alloy, a conductive polymer, graphene, carbon nanotubes, nanowires or combinations thereof. They may be patterned by any suitable method such as ink jet printing, flexography, photolithography, thermal transfer, vapor deposited through a shadow mask and the like. For example, the contact pads, conductive traces and electrode pads are made of Au and patterned by lift-off photolithography.

FIG. 15C illustrates the formation of second intermediate structure 302 having patterned insulating layer 384 (illustrated as 384a for bioelectronic device 391a) provided over the conductive traces 382 leaving open the electrode pads 383 and the contact pads 381. The tip of shank portion 398 corresponding to bioelectronic device 391a of the second intermediate structure 302 is highlighted by circle B in FIG. 15C and a magnified plan view is shown in FIG. 15D. Patterned insulating layer may be any of the polymers previously mentioned, and is preferably biocompatible. In an embodiment, the patterned insulating layer is a fluoropolymer, preferably formed from a photosensitive fluoropolymer.

One or more of the electrode pads may optionally be further modified. In an embodiment, the modification is done after forming the patterned insulating layer, but in another embodiment, it is done before. Choice of the modifying material depends upon the function of the particular electrode pad. For example, a conductive polymer such as PEDOT:PSS, may be provided over the electrode pad to form a biosensor electrode as described previously for conductive polymer 140. In an embodiment, a modifying material may be patterned by photolithography. Alternatively, a modifying material may be ink jetted over the electrode pad and into a well structure formed by the patterned insulating layer around the electrode pad. Since the electrode pads are addressable, a modifying material may be provided by electrochemical methods, e.g., by electroplating, electropolymerization, electrophoretic deposition, anodization of the electrode pad surface or the like. Alternatively, the electrode pad may be made of a material that selectively binds another chemical to form a modifying material layer. For example, a gold electrode pad can bind thiol-containing compounds to form a self-assembled monolayer of modifying material upon simple dip-coating. A modifying material may be doctor-bladed into wells. In FIG. 15D, one of the electrode pads of bioelectronic device 391a has been modified with an overcoat of conductive polymer (modifying material) 385a. To further illustrate the device at this point, FIG. 15E is a cross-sectional view taken along cut line C-C drawn in FIG. 15D. In an embodiment, the patterning of modifying material 385a is done prior to forming the patterned insulating layer 384a, and the patterned insulating layer may optionally extend over the top edges of modifying material 385a.

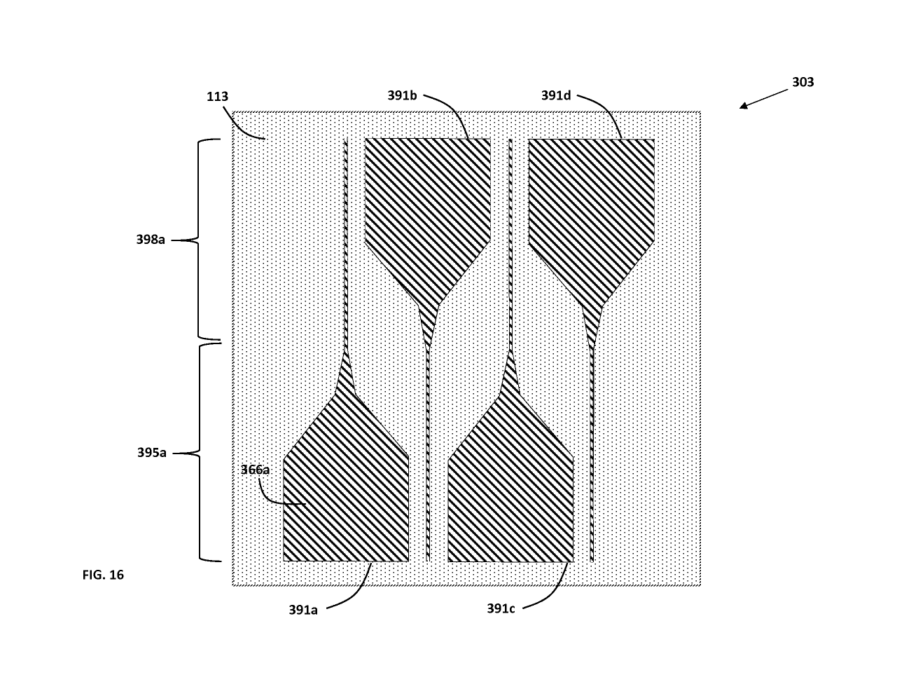

Next, a layer of bioresorbable material is provided over the second intermediate structure (which is includes the flexible substructure), and as described previously, a layer of fluoropolymer is provided in a first pattern over the layer of bioresorbable material and a second pattern of non-covered bioresorbable material is selectively removed using the fluoropolymer as an etch barrier. FIG. 16 shows a plan view of this third intermediate structure 303. The layer of patterned bioresorbable material is hidden beneath the first pattern of fluoropolymer 366 in this view, but the pattern of uncovered substructure 113 corresponding to the second pattern is shown.

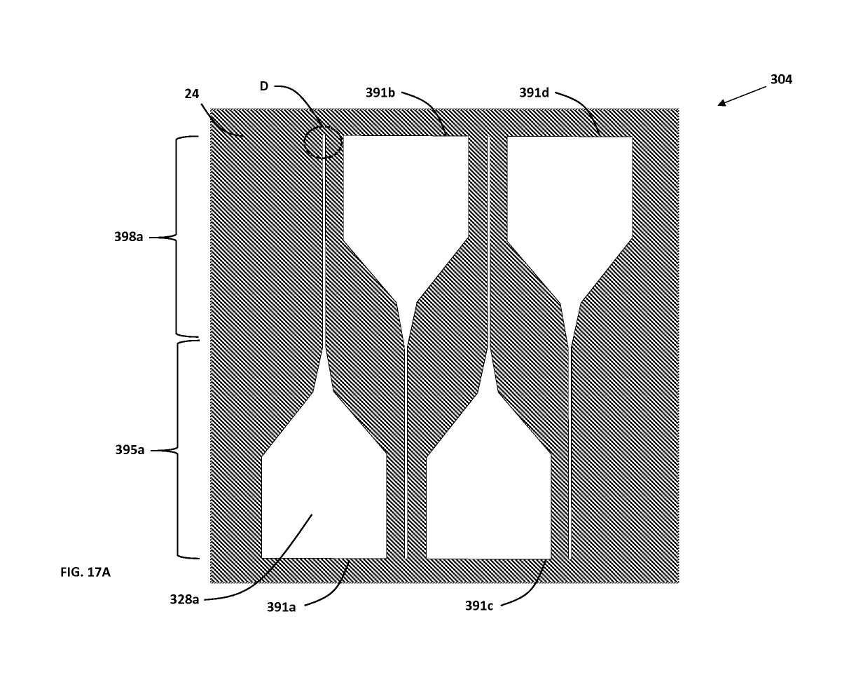

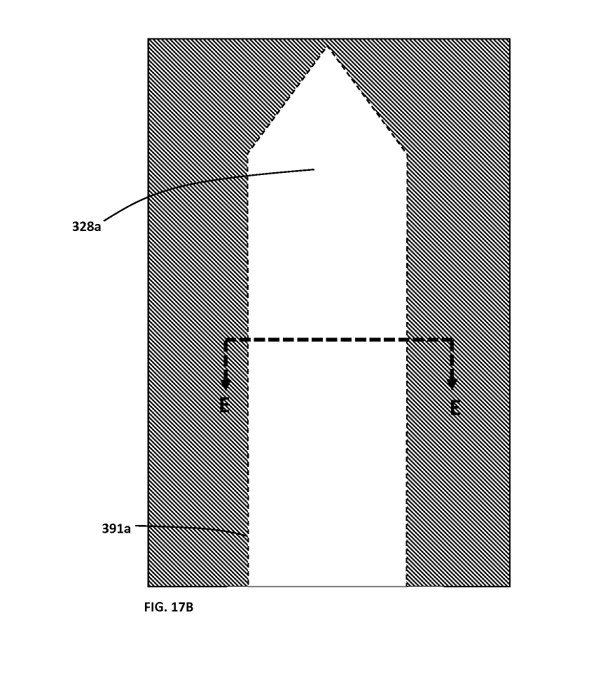

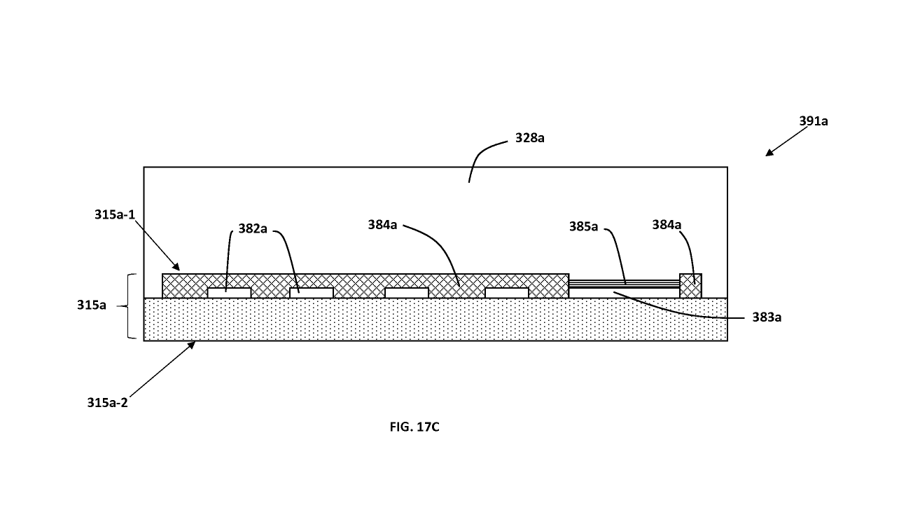

In an embodiment, the pattern of uncovered substructure is then selectively removed down to the release layer 24 using the first pattern of fluoropolymer, the patterned bioresorbable material or both as an etch barrier. In the embodiment shown in FIG. 17A, release layer 24 was not removed, but in alternative embodiment, the release layer 24 may instead be etched in areas not covered by the first pattern of fluoropolymer. In this embodiment, the first pattern of fluoropolymer is stripped following the selective removal of the substructure, thereby forming the array 304 of patterned bioelectronic devices 391a, 391b, 391c and 391d, each having a layer of patterned bioresorbable material 328 and a patterned substructure 315a under the bioresorbable material. A plan view of the array is shown as FIG. 17A. The tip of shank portion 398 corresponding to bioelectronic device 391a is highlighted by circle D in FIG. 17A and a magnified plan view is shown in FIG. 17B. To further illustrate the device, FIG. 17C is a cross-sectional view taken along cutline E-E drawn in FIG. 17B and after removal from the carrier substrate (using methods described previously). Patterned bioelectronic device 391a includes patterned flexible substructure 315a having a first side (315a-1) and a second side (315a-2). The bioelectronic feature forms a portion of the first side. The patterned bioresorbable material 328a is provided only on the first side and there is substantially no bioresorbable material on the second side. By “substantially no bioresorbable material”, it is meant that if any bioresorbable material is present on the second side, its total mass is less than 10% of the mass of patterned bioresorbable material 328a on the first side. In this embodiment, the bioresorbable material is provided in direct contact with the bioelectronic feature.

In the embodiment shown in FIG. 17A, the contact pads 381 are covered with bioresorbable material. Contact to electronic circuitry can optionally be made using connectors capable of penetrating the bioresorbable material layer. Alternatively, the patterned bioresorbable material layer can undergo a second patterning, e.g., using the patterned fluoropolymer method described above, to selectively remove bioresorbable material from the contact pad area.

The shank portion of the probe may optionally be inserted into living tissue and each electrode pad (bioelectronic feature) may be electronically addressed to serve some function. In some non-limiting examples, an electrode pad may serve to inject charge into the tissue (bio-stimulator) or it may serve to measure a biological environment condition (bio-sensor). Each probe in the above embodiment has five electrode pads, so each electrode pad may optionally serve different functions.

Embodiments like the one above can have several advantages. First, the presence of the bioresorbable material may enhance the mechanical strength of the flexible substructure, to aid handling or assist in insertion into target tissue. By providing the bioresorbable in the pattern of the flexible substructure (at least the bioactive portion), and only on one side, the amount of bioresorbable material that living tissue may need to resorb is reduced, thereby lowering stresses such resorption may have on a biological target. In addition, providing bioresorbable material over the flexible substructure on the same side as the bioelectronic feature, the bioelectronic feature is protected from damage during handling or insertion.

In the above embodiment, the bioresorbable material covers certain bioelectronic features of the flexible substructure. As discussed, there can be advantages to such a structure. In some embodiments, however, it may be useful for the bioelectronic feature to be in immediate, direct contact with the bio-environment without an intervening bioresorbable material. In an alternative embodiment, a bioelectronic device may be provided with a patterned bioresorbable material that does not cover the bioelectronic feature.

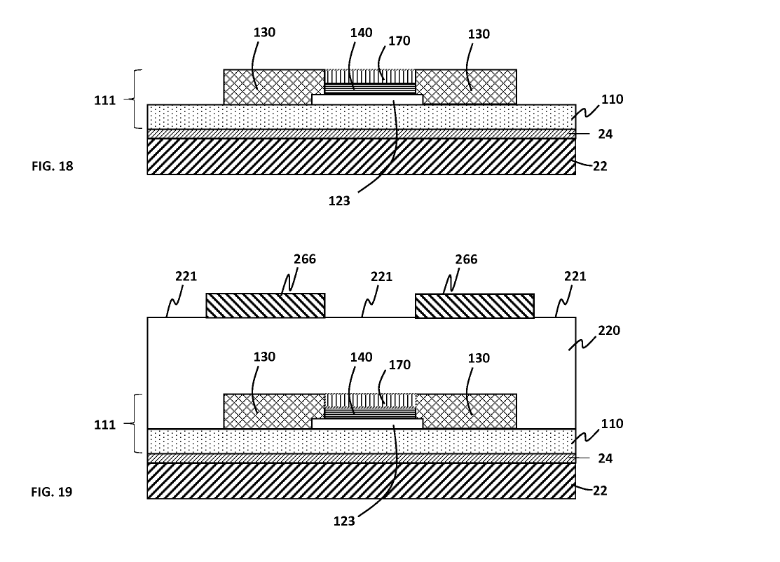

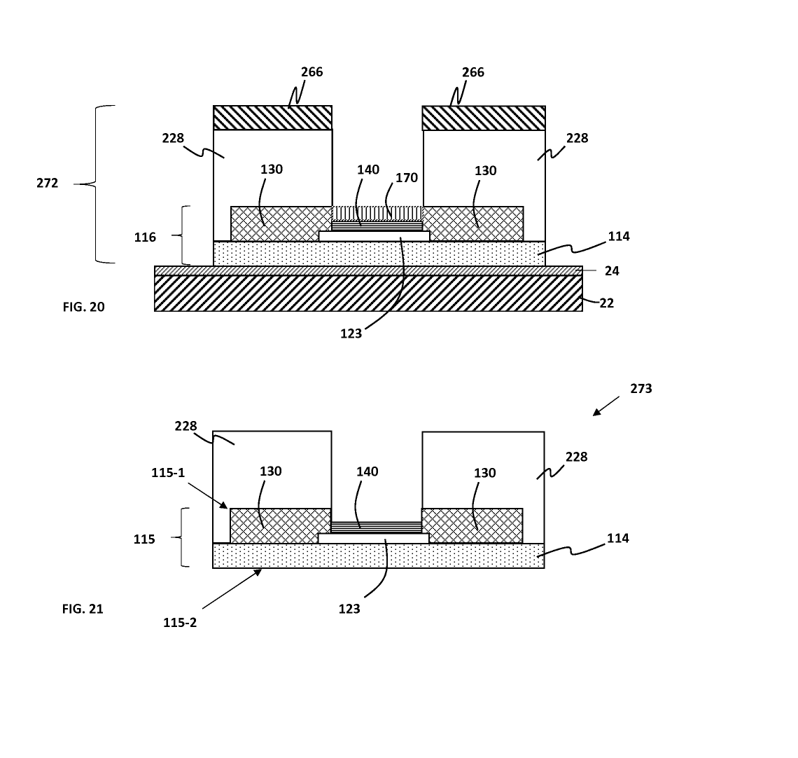

One method is illustrated in FIGS. 18-21, starting with the structure of FIG. 7 previously described. In FIG. 18, a protection layer 170 is provided over the conductive polymer layer 140 to form a protected bioelectronic feature and protected flexible substructure 111. In a preferred embodiment, the protection layer is a fluoropolymer that is resistant to the etch steps used to remove bioresorbable material and other areas of the flexible substructure. If the bioelectronic feature is inherently resistant to these etch steps, then the protection layer is not necessary. In FIG. 19, a layer of fluoropolymer 266 is provided in a first pattern over the layer of bioresorbable material 220, thereby forming a complementary second pattern of non-covered bioresorbable material, 221. In this embodiment the first pattern of fluoropolymer 266 does not cover the protected bioelectronic feature. Etching of the bioresorbable material and flexible substructure is performed as previously described to form patterned bioresorbable material 228 and patterned bioelectronic device 272, as shown in FIG. 20. Although the flexible support 110 is susceptible to etching, thereby forming patterned flexible support 114 and patterned, protected flexible substructure 116, the protection layer 170 prevented etching of the bioelectronic feature. The protection layer 170 may block the functionality of the bioelectronic feature, so it is preferably removed after etching. In an embodiment, a fluorinated stripping solvent is used to remove both the patterned layer of fluoropolymer 266 and the protection layer 170 in a common step, to form patterned bioelectronic device 273 having patterned substructure 115 shown in FIG. 21 after removal from the carrier substrate (using methods described previously). Patterned flexible substructure 115 has a first side (115-1) and a second side (115-2). The bioelectronic feature forms a portion of the first side. The patterned bioresorbable material 228 is provided only on the first side, but includes an opening aligned with the conductive polymer 140 (bioelectronic feature), so as not to cover or be in contact with the bioelectronic feature. In this embodiment, there is substantially no bioresorbable material on the second side. This approach may be used to make an array of bioelectronic devices wherein at least one of the bioelectronic features is not covered by bioresorbable material.

Embodiments like the one in FIG. 21 have similar advantages as discussed previously. Such bioresorbable material structures may provide less protection of the bioelectronic feature than when the bioresorbable material is directly covering it, but allows more immediate direct contact between the bioelectronic feature and the biological environment, which may be advantageous in some applications.

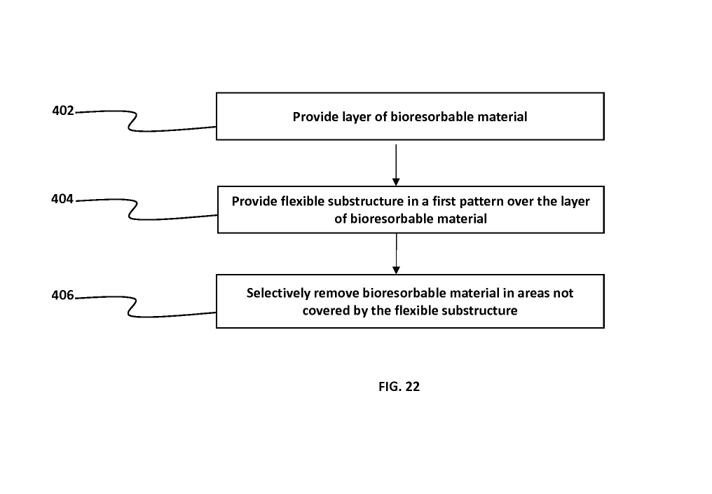

In another embodiment, a flexible substructure having a bioelectronic feature is provided over the bioresorbable material. A flow diagram is shown in FIG. 22, and includes the step 402 of providing a layer of bioresorbable material, the step 404 of providing a flexible substructure in a first pattern over the bioresorbable material, and the step 406 of selectively removing bioresorbable material in areas not covered by the flexible substructure. Each step is discussed in more detail, below.

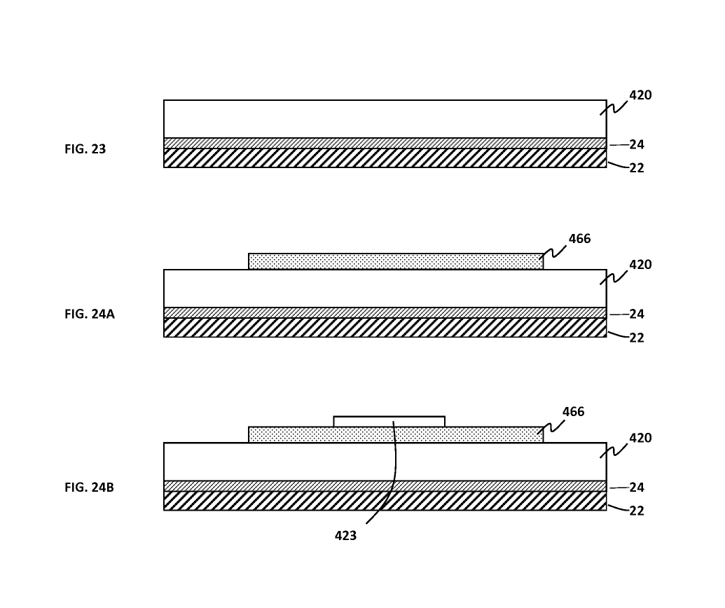

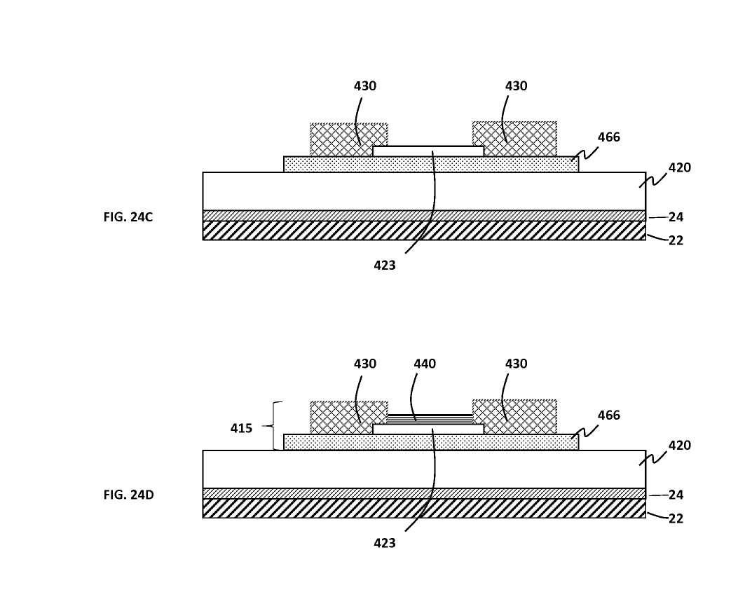

Turning to an embodiment shown in FIG. 23, a layer of bioresorbable material 420 is provided over an optional carrier substrate 22 along with an optional intervening release layer 24. Materials and methods related to these features have already been described above, e.g., see the discussion regarding FIG. 2. Next, as shown in FIGS. 24A-24D, a flexible substructure 415 is provided in a first pattern over the layer of bioresorbable material 420. In FIG. 24A, a patterned fluoropolymer layer 466 is provided over the layer of bioresorbable material. Methods for patterning a fluoropolymer layer have been described above. Whichever method is used needs to be compatible with the layer of bioresorbable material. In the present embodiment, the fluoropolymer is formed from a photosensitive, cross-linking fluoropolymer and functions as a flexible support over which a bioelectronic feature is formed. In an embodiment, the patterned fluoropolymer layer 466 is selected so that it is not readily soluble in most fluorinated or other solvents. In an embodiment, the patterned fluoropolymer is a photosensitive cross-linking fluoropolymer. In FIG. 24B a conductive metal electrode 423 (a bioelectronic feature) is provided over the patterned fluoropolymer layer 466. Metal electrode 423 may be formed by ink jet deposition, flexographic printing, vapor deposition through a shadow mask, photolithography, or other methods, so long as they do not deleteriously interact with the layer of bioresorbable material. For example, the metal electrode may be a bilayer of Au/Ti formed by lift-off photolithography using a photosensitive fluoropolymer that is deposited, developed and stripped using fluorinated solvents that do not significantly interact with the bioresorbable material. In FIG. 24C, patterned electrical insulator layer 430 is provided in a pattern that covers edge portion of metal electrode 423 and some of patterned fluoropolymer 466. Although not shown, the insulator layer may also cover wiring, as previously described in earlier embodiments of flexible substructures. The insulator layer may be made from the same materials and by the same methods as previously disclosed with respect to patterned electrical insulator layer 130, except that the insulator material and method must be compatible with the layer of bioresorbable material. In an embodiment, the insulator layer 430 is provided using a negative tone, cross-linking photosensitive fluoropolymer that is exposed through a photomask and developed using a fluorinated solvent. As mentioned earlier, the metal electrode may optionally be modified in some way. In FIG. 24D, a conductive polymer 440 may be provided in a manner as discussed for conductive polymer 140, above. In this embodiment, the deposition method should be selected so that it is compatible with the layer of bioresorbable material 420. For example, the conductive polymer 440 may be deposited by ink jet. Flexible substructure 415 has a first pattern that, in this embodiment, is defined by the patterned fluoropolymer 466. In another embodiment, the insulator layer 430 may be chosen to extend beyond the edge of patterned fluoropolymer 466, and the first pattern would comprise the combined layers of 430 and 466. In other words, when observing from above, the first pattern is defined by the layers that laterally extend the furthest.

In FIG. 24E, a protection layer 470 is provided over the conductive polymer layer 140 to form a protected bioelectronic feature and protected flexible substructure 416, also having a first pattern. In a preferred embodiment, the protection layer is a fluoropolymer that is resistant to the etch steps used to remove bioresorbable material. If the bioelectronic feature is inherently resistant to these etch steps, then the protection layer is not necessary.

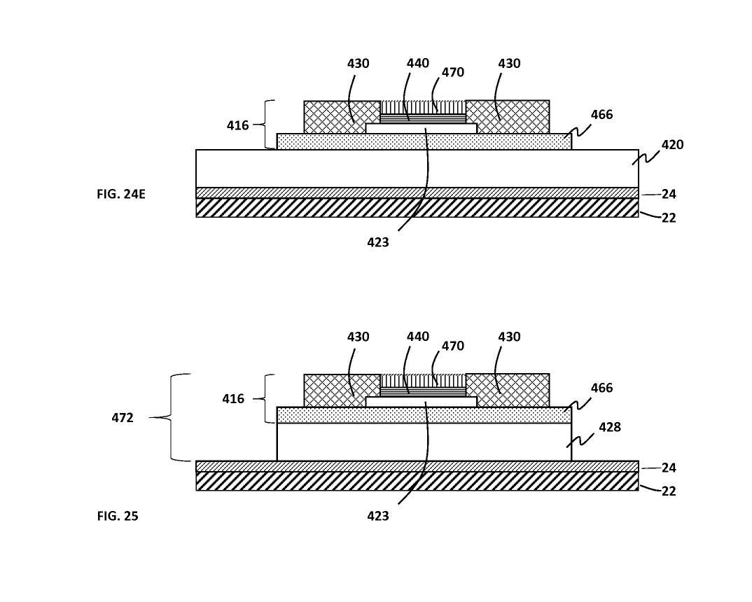

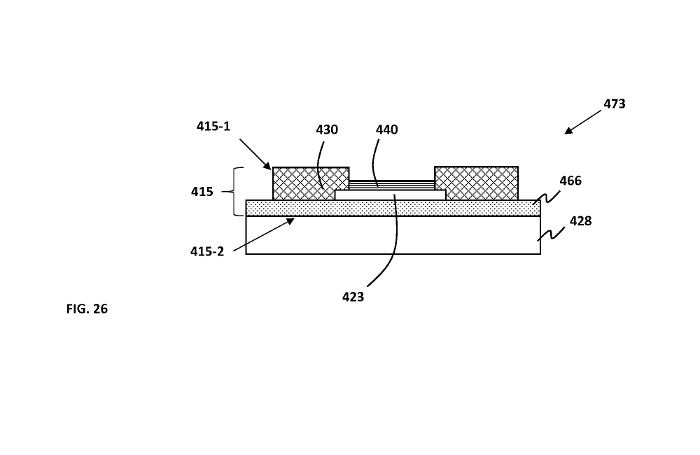

Turning to FIG. 25, the structure from FIG. 24E is subjected to an etching environment, e.g., a plasma etch or a solvent etch, that selectively removes bioresorbable material not covered by the first pattern of flexible substructure, thereby forming patterned bioresorbable material 428 and patterned bioelectronic device 472. In this embodiment, the protected flexible substructure 416 acts as an etch barrier. Because the protection layer 470 may interfere with the operation of the patterned bioelectronic device, it is preferably removed after etching the bioresorbable material, thereby forming patterned bioelectronic device 473 having patterned flexible substructure 415, as shown in FIG. 26 after removal from the carrier substrate (using methods described previously). An array of such patterned bioelectronic devices may be fabricated. Patterned flexible substructure 415 has a first side (415-1) and a second side (415-2). The bioelectronic feature forms a portion of the first side. The patterned bioresorbable material 428 is provided only on the second side and substantially no bioresorbable material is provided on the first side. Embodiments like the one shown in FIG. 26 have similar advantages to the structures previously described except there is no additional protection of the bioelectronic feature. On the other hand, such structures allow immediate, direct contact between the bioelectronic feature and the biological environment, which may be advantageous in some applications.

As mentioned above, certain processing steps of the present disclosure may include the use of a fluorinated solvent. When fluorinated solvents are used, they may be used in some embodiments as mixtures or solutions with non-fluorinated materials, but typically such mixtures include at least 50% by volume of a fluorinated solvent, preferably at least 90% by volume. Depending on the particular material set and solvation needs of the process, the fluorinated solvent may be selected from a broad range of materials such as chlorofluorocarbons (CFCs), hydrochlorofluorocarbons (HCFCs), hydrofluorocarbons (HFCs), perfluorocarbons (PFCs), hydrofluoroethers (HFEs), perfluoroethers, perfluoroamines, trifluoromethyl-substituted aromatic solvents, fluoroketones and the like.

Particularly useful fluorinated solvents include those that are perfluorinated or highly fluorinated liquids at room temperature, which are immiscible with water and most (but not necessarily all) organic solvents. Among those solvents, hydrofluoroethers (HFEs) are well known to be highly environmentally friendly, “green” solvents. HFEs, including segregated HFEs, are preferred solvents because they are non-flammable, have zero ozone-depletion potential, lower global warming potential than PFCs and show very low toxicity to humans.

Examples of readily available HFEs and isomeric mixtures of HFEs include, but are not limited to, an isomeric mixture of methyl nonafluorobutyl ether and methyl nonafluoroisobutyl ether (HFE-7100), an isomeric mixture of ethyl nonafluorobutyl ether and ethyl nonafluoroisobutyl ether (HFE-7200 aka Novec™ 7200), 3-ethoxy-1,1,1,2,3,4,4,5,5,6,6,6-dodecafluoro-2-trifluoromethyl-hexane (HFE-7500 aka Novec™ 7500), 1,1,1,2,3,3-hexafluoro-4-(1,1,2,3,3,3,-hexafluoropropoxy)-pentane (HFE-7600 aka Novec™ 7600), 1-methoxyheptafluoropropane (HFE-7000), 1,1,1,2,2,3,4,5,5,5-decafluoro-3-methoxy-4-trifluoromethylpentane (HFE-7300 aka Novec™ 7300), 1,3-(1,1,2,2-tetrafluoroethoxyl)benzene (HFE-978m), 1,2-(1,1,2,2-tetrafluoroethoxyl)ethane (HFE-578E), 1,1,2,2-tetrafluoroethyl-1H,1H,5H-octafluoropentyl ether (HFE-6512), 1,1,2,2-tetrafluoroethyl-2,2,2-trifluoroethyl ether (HFE-347E), 1,1,2,2-tetrafluoroethyl-2,2,3,3-tetrafluoropropyl ether (HFE-458E), 2,3,3,4,4-pentafluorotetrahydro-5-methoxy-2,5-bis[1,2,2,2-tetrafluoro-1-(trifluoromethyl)ethyl]-furan (HFE-7700 aka Novec™ 7700) and 1,1,1,2,2,3,3,4,4,5,5,6,6-tridecafluorooctane-propyl ether (TE60-C3).

Mixtures of fluorinated solvents may optionally be used, e.g., as disclosed in U.S. patent application Ser. Nos. 14/260,666 and 14/260,705, the entire contents of which are incorporated by reference herein.

The term “fluoropolymer” herein includes not only high molecular weight, long chain fluorinated materials, but also lower molecular weight oligomers, macrocyclic compounds such as fluorinated calixarene derivatives and other highly fluorinated hydrocarbons having at least 15 carbon atoms. In an embodiment, the molecular weight of the fluoropolymer is at least 750. In an embodiment, the fluoropolymer is soluble in one or more fluorinated solvents. Fluoropolymers preferably have a total fluorine content by weight in a range of 15% to 75%, or alternatively 30% to 75%, or alternatively 30% to 55%.

When the fluoropolymer is provided as a layer that is not inherently photosensitive (not directly photopatternable, such as described in FIG. 5), the fluorine content by weight is preferably in a range of 40% to 75%. Some non-limiting coatable examples of such polymers include Teflon AF (copolymer of tetrafluoroethylene with 2,2′-bis(trifluoromethyl)-4,5-difluoro-1,3-dioxole) and Cytop (a cyclic polymer formed from F₂C═CFCF₂OCF═CF₂). In an embodiment, the non-inherently photosensitive fluoropolymer is a copolymer comprising a fluorine-containing group (see below for examples) and a non-photosensitive functional group. The non-photosensitive functional group may improve film adhesion, improve coatability, adjust dissolution rate, absorb light, improve etch resistance and the like. In an embodiment, the non-photosensitive functional group is a non-fluorine-containing aromatic or aliphatic hydrocarbon that may optionally be substituted, for example, with oxygen-containing groups such as ethers, alcohols, esters, and carboxylic acids.

Photosensitive fluoropolymers can be provided, e.g., by coating a photosensitive fluoropolymer composition (also referred to herein as a fluorinated photopolymer composition) that includes a fluorinated solvent, a fluorinated photopolymer material, and optionally additional materials such as sensitizing dyes, photo-acid generator compounds, stabilizers, and the like. In an embodiment, the fluorinated photopolymer material includes a copolymer comprising at least two distinct repeating units, including a first repeating unit having a fluorine-containing group and a second repeating unit having a solubility-altering reactive group. In an embodiment using a fluorinated photopolymer that is a copolymer, the copolymer has a total fluorine content of at least 10%, preferably at least 15%. In an embodiment, the total fluorine content is in a range of 15% to 60%, alternatively 30 to 60%, or alternatively 35 to 55%. The copolymer is suitably a random copolymer, but other copolymer types may be used, e.g., block copolymers, alternating copolymers, and periodic copolymers. The term “repeating unit” herein is used broadly herein and simply means that there is more than one unit. The term is not intended to convey that there is necessarily any particular order or structure with respect to the other repeating units unless specified otherwise. When a repeating unit represents a low mole % of the combined repeating units, there may be only one such unit on a polymer chain. The copolymer may be optionally blended with one or more other polymers, preferably other fluorine-containing polymers. The fluoropolymer may optionally be branched, which may in certain embodiments enable lower fluorine content, faster development and stripping rates, or incorporation of groups that otherwise may have low solubility in a fluorinated polymer. Non-limiting examples of photosensitive fluoropolymer compositions are described in US Patent Publication 2011/0159252, U.S. patent application Ser. Nos. 14/113,408, 14/291,692, 14/335,476, U.S. Provisional Patent Application Nos. 61/990,966, and 61/937,122, the contents of which are incorporated by reference.

In an embodiment, at least one of the specified repeat units is formed via a post-polymerization reaction. In this embodiment, an intermediate polymer (a precursor to the desired copolymer) is first prepared, said intermediate polymer comprising suitably reactive functional groups for forming one of more of the specified repeat units. For example, an intermediate polymer containing pendant carboxylic acid moieties can be reacted with a fluorinated alcohol compound in an esterification reaction to produce the specified fluorinated repeating unit. Similarly, a precursor polymer containing an alcohol can be reacted with a suitably derivatized glycidyl moiety to form an acid-catalyzed cross-linkable (epoxy-containing) repeating unit as the solubility-altering reactive group. In another example, a polymer containing a suitable leaving group such as primary halide can be reacted with an appropriate compound bearing a phenol moiety to form the desired repeat unit via an etherification reaction. In addition to simple condensation reactions such as esterification and amidation, and simple displacement reactions such as etherification, a variety of other covalent-bond forming reactions well-known to practitioners skilled in the art of organic synthesis can be used to form any of the specified repeat units. Examples include palladium-catalyzed coupling reactions, “click” reactions, addition to multiple bond reactions, Wittig reactions, reactions of acid halides with suitable nucleophiles, and the like.

In an alternative embodiment, the first and second repeating units of the copolymer are formed directly by polymerization of two (or more) appropriate monomers, rather than by attachment to an intermediate polymer. Although many of the embodiments below refer to polymerizable monomers, analogous structures and ranges are contemplated wherein one or more of the first and second repeating units are formed by attachment of the relevant group to an intermediate polymer as described above.

In an embodiment, the fluorinated photopolymer material includes a copolymer formed at least from a first monomer having a fluorine-containing group and a second monomer having a solubility-altering reactive group. Additional monomers may optionally be incorporated into the copolymer. The first monomer is one capable of being copolymerized with the second monomer and has at least one fluorine-containing group. In an embodiment, at least 70% of the fluorine content of the copolymer (by weight) is derived from the first monomer. In another embodiment, at least 85% of the fluorine content of the copolymer (by weight) is derived from the first monomer.

The first monomer includes a polymerizable group and a fluorine-containing group. Some non-limiting examples of useful polymerizable groups include acrylates (e.g. acrylate, methacrylate, cyanoacrylate and the like), acrylamides, vinylenes (e.g., styrenes), vinyl ethers and vinyl esters. The fluorine-containing group of the first monomer or the first repeating unit is preferably an alkyl or aryl group that may optionally be further substituted with chemical moieties other than fluorine, e.g., chlorine, a cyano group, or a substituted or unsubstituted alkyl, alkoxy, alkylthio, aryl, aryloxy, amino, alkanoate, benzoate, alkyl ester, aryl ester, alkanone, sulfonamide or monovalent heterocyclic group, or any other substituent that a skilled worker would readily contemplate that would not adversely affect the performance of the fluorinated photopolymer. Throughout this disclosure, unless otherwise specified, any use of the term alkyl includes straight-chain, branched and cyclo alkyls. In an embodiment, the first monomer does not contain protic or charged substituents, such as hydroxy, carboxylic acid, sulfonic acid or the like.

In an embodiment, the first monomer has a structure according to formula (1):

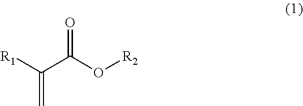

In formula (1), R₁ represents a hydrogen atom, a cyano group, a methyl group or an ethyl group. R₂ represents a fluorine-containing group, in particular, a substituted or unsubstituted alkyl group having at least 5 fluorine atoms, preferably at least 10 fluorine atoms. In an embodiment, the alkyl group is a cyclic or non-cyclic hydrofluorocarbon or hydrofluoroether having at least as many fluorine atoms as carbon atoms. In a preferred embodiment R₂ represents a perfluorinated alkyl or a 1H,1H,2H,2H-perfluorinated alkyl having at least 4 carbon atoms. An example of the latter is 1H,1H,2H,2H-perfluorooctyl methacrylate (“FOMA”).

A combination of multiple first monomers or first repeating units having different fluorine-containing groups may be used. The total mole ratio of first monomers relative to all of the monomers of the copolymer may be in a range of 5 to 80%, or alternatively 10 to 70%, or alternatively 20 to 60%.

The second monomer is one capable of being copolymerized with the first monomer. The second monomer includes a polymerizable group and a solubility-altering reactive group. Some non-limiting examples of useful polymerizable groups include those described for the first monomer.

In an embodiment, the solubility-altering reactive group of the second monomer or second repeating unit is an acid-forming precursor group. Upon exposure to light, the acid-forming precursor group generates a polymer-bound acid group, e.g., a carboxylic or sulfonic acid. This can drastically change its solubility relative to the unexposed regions thereby allowing development of an image with the appropriate solvent. In an embodiment, the developing agent includes a fluorinated solvent that selectively dissolves unexposed areas. In an alternative embodiment, the developing agent includes a polar solvent that selectively dissolves the exposed areas. In an embodiment, a carboxylic acid-forming precursor is provided from a monomer in a weight percentage range of 4 to 40% relative to the copolymer, or alternatively in a weight percentage range of 10 to 30%.

One class of acid-forming precursor groups includes the non-chemically amplified type (i.e., non-acid catalyzed). An example of a second monomer with such a group is 2-nitrobenzyl methacrylate. The non-chemically amplified precursor group may directly absorb light to initiate de-protection of the acid-forming groups. Alternatively, a sensitizing dye may be added to the composition whereby the sensitizing dye absorbs light and forms an excited state capable of directly sensitizing or otherwise initiating the de-protection of acid-forming precursor groups. The sensitizing dye may be added as a small molecule or it may be attached or otherwise incorporated as part of the copolymer. Unlike chemically amplified formulations that rely on generation of an acid (see below), non-chemically amplified photopolymers may sometimes be preferred when a photopolymer is used in contact with an acid-sensitive or acid-containing material.

A second class of acid-forming precursor groups includes the chemically amplified type. This typically requires addition of a photo-acid generator (PAG) to the photopolymer composition, e.g., as a small molecule additive to the solution. The PAG may function by directly absorbing radiation (e.g. UV light) to cause decomposition of the PAG and release an acid. Alternatively, a sensitizing dye may be added to the composition whereby the sensitizing dye absorbs radiation and forms an excited state capable of reacting with a PAG to generate an acid. The sensitizing dye may be added as a small molecule, e.g., as disclosed in U.S. patent application Ser. No. 14/335,476, which is incorporated herein by reference. The sensitizing dye may be attached to or otherwise incorporated as part of the copolymer, e.g., as disclosed in U.S. patent application Ser. Nos. 14/291,692 and 14/291,767, which are incorporated herein by reference. In an embodiment, the sensitizing dye (either small molecule or attached) is fluorinated. In an embodiment, the sensitizing dye may be provided in a range of 0.5 to 10% by weight relative to the total copolymer weight. The photochemically generated acid catalyzes the de-protection of acid-labile protecting groups of the acid-forming precursor. In some embodiments, chemically amplified photopolymers can be particularly desirable since they enable the exposing step to be performed through the application of relatively low energy UV light exposure. This is advantageous since some active organic materials useful in applications to which the present disclosure pertains may decompose in the presence of UV light, and therefore, reduction of the energy during this step permits the photopolymer to be exposed without causing significant photolytic damage to underlying active organic layers. Also, reduced light exposure times improve the manufacturing throughput of the desired devices.

Examples of acid-forming precursor groups that yield a carboxylic acid include, but are not limited to: A) esters capable of forming, or rearranging to, a tertiary cation, e.g., t-butyl ester, t-amyl ester, 2-methyl-2-adamantyl ester, 1-ethylcyclopentyl ester, and 1-ethylcyclohexyl ester; B) esters of lactone, e.g., γ-butyrolactone-3-yl, γ-butyrolactone-2-yl, mevalonic lactone, 3-methyl-y-butyrolactone-3-yl, 3-tetrahydrofuranyl, and 3-oxocyclohexyl; C) acetal esters, e.g., 2-tetrahydropyranyl, 2-tetrahydrofuranyl, and 2,3-propylenecarbonate-1-yl; D) beta-cyclic ketone esters, E) alpha-cyclic ether esters; and F) MEEMA (methoxy ethoxy ethyl methacrylate) and other esters which are easily hydrolyzable because of anchimeric assistance. In an embodiment, the second monomer comprises an acrylate-based polymerizable group and a tertiary alkyl ester acid-forming precursor group, e.g., t-butyl methacrylate (“TBMA”) or 1-ethylcyclopentyl methacrylate (“ECPMA”).

In an embodiment, the solubility-altering reactive group is an hydroxyl-forming precursor group (also referred to herein as an “alcohol-forming precursor group”). The hydroxyl-forming precursor includes an acid-labile protecting group and the photopolymer composition typically includes a PAG compound and operates as a “chemically amplified” type of system. Upon exposure to light, the PAG generates an acid (either directly or via a sensitizing dye as described above), which in turn, catalyzes the deprotection of the hydroxyl-forming precursor group, thereby forming a polymer-bound alcohol (hydroxyl group). This significantly changes its solubility relative to the unexposed regions thereby allowing development of an image with the appropriate solvent (typically fluorinated). In an embodiment, the developing agent includes a fluorinated solvent that selectively dissolves unexposed areas. In an alternative embodiment, the developing agent includes a polar solvent that selectively dissolves the exposed areas. In an embodiment, an hydroxyl-forming precursor is provided from a monomer in a weight percentage range of 4 to 40% relative to the copolymer.

In an embodiment, the hydroxyl-forming precursor has a structure according to formula (2):

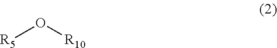

wherein R₅ is a carbon atom that forms part of the second repeating unit or second monomer, and R₁₀ is an acid-labile protecting group. Non-limiting examples of useful acid-labile protecting groups include those of formula (AL-1), acetal groups of the formula (AL-2), tertiary alkyl groups of the formula (AL-3) and silane groups of the formula (AL-4).

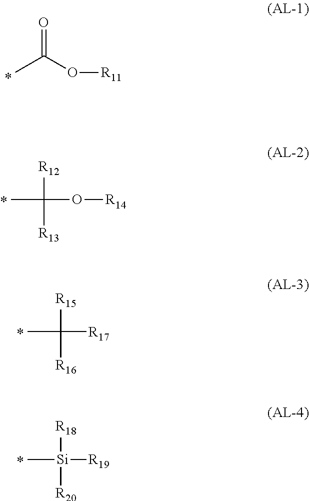

In formula (AL-1), R₁₁ is a monovalent hydrocarbon group, typically a straight, branched or cyclic alkyl group, of 1 to 20 carbon atoms that may optionally be substituted with groups that a skilled worker would readily contemplate would not adversely affect the performance of the precursor. In an embodiment, R₁₁ may be a tertiary alkyl group. Some representative examples of formula (AL-1) include:

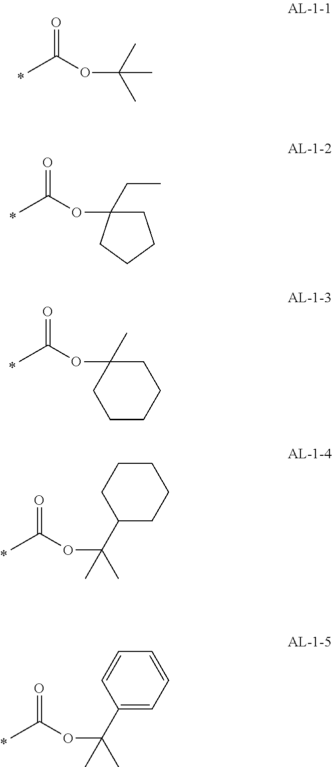

In formula (AL-2), R₁₄ is a monovalent hydrocarbon group, typically a straight, branched or cyclic alkyl group, of 1 to 20 carbon atoms that may optionally be substituted. R₁₂ and R₁₃ are independently selected hydrogen or a monovalent hydrocarbon group, typically a straight, branched or cyclic alkyl group, of 1 to 20 carbon atoms that may optionally be substituted. Some representative examples of formula (AL-2) include:

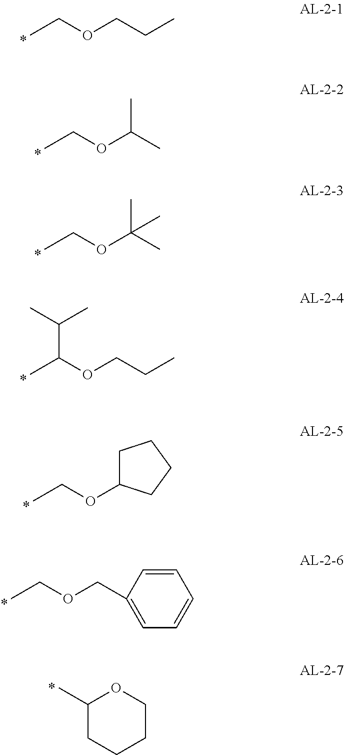

In formula (AL-3), R₁₅, R₁₆, and R₁₇ represent an independently selected a monovalent hydrocarbon group, typically a straight, branched or cyclic alkyl group, of 1 to 20 carbon atoms that may optionally be substituted. Some representative examples of formula (AL-3) include:

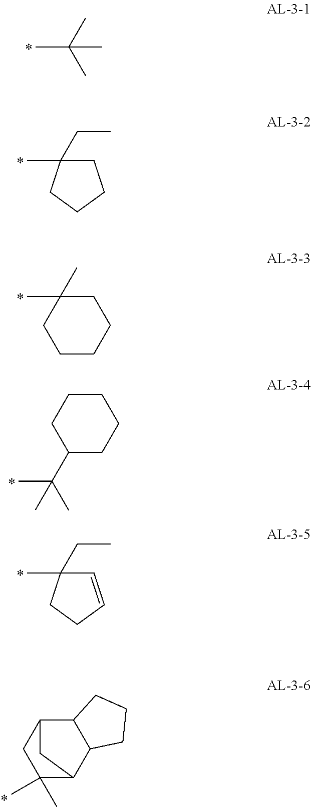

In formula (AL-4), R₁₈, R₁₉ and R₂₀ are independently selected hydrocarbon groups, typically a straight, branched or cyclic alkyl group, of 1 to 20 carbon atoms that may optionally be substituted.

The descriptions of the above acid-labile protecting groups for formulae (AL-2), (AL-3) and (AL-4) have been described in the context of hydroxyl-forming precursors. These same acid-labile protecting groups, when attached instead to a carboxylate group, may also be used to make some of the acid-forming precursor groups described earlier.

In an embodiment, the solubility-altering reactive group is a cross-linkable group, e.g., an acid-catalyzed cross-linkable group or a photo cross-linkable (non-acid catalyzed) group. Photo cross-linkable groups typically have at least one double bond so that when the group forms an excited state (either by direct absorption of light or by excited state transfer from a sensitizing dye), sets of double bonds from adjacent polymer chains crosslink. In an embodiment, the photo cross-linkable group (not catalyzed) comprises a cinnamate that may optionally further include fluorine-containing substituents, as described in U.S. Provisional Application No. 61/937,122, the contents of which are incorporated herein. Some non-limiting examples of polymerizable monomers including such cinnamates are shown below

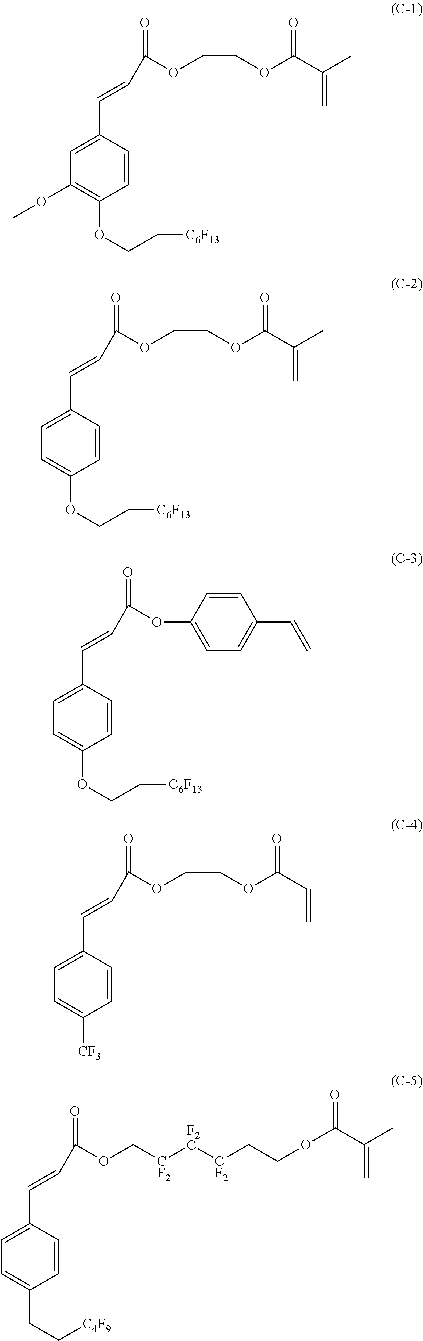

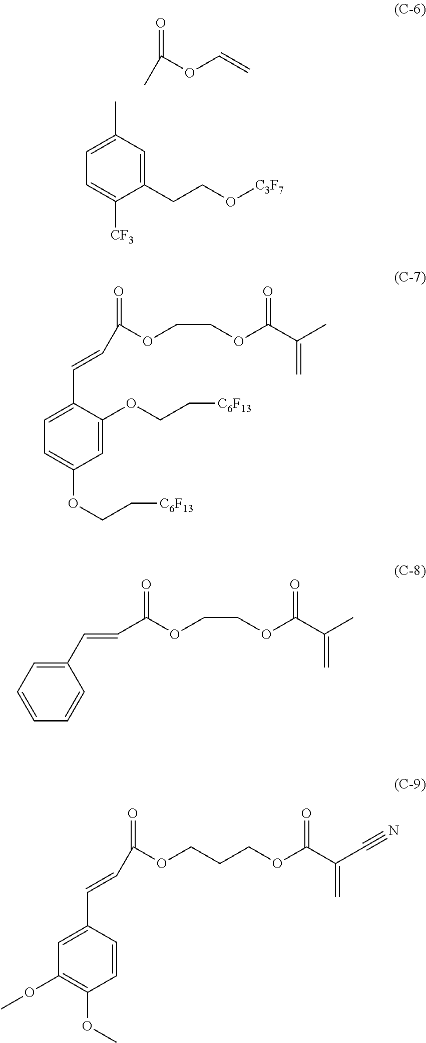

Compositions comprising such materials may optionally further include a sensitizing dye. Some non-limiting examples of useful sensitizing dyes for cinnamate cross-linking groups include diaryl ketones (e.g., benzophenones), arylalkyl ketones (e.g., acetophenones), diaryl butadienes, diaryl diketones (e.g. benzils), xanthones, thioxanthones, naphthalenes, anthracenes, benzanthrone, phenanthrenes, crysens, anthrones, 5-nitroacenapthene, 4-nitroaniline, 3-nitrofluorene, 4-nitromethylaniline, 4-nitrobiphenyl, picramide, 4-nitro-2,6-dichlorodimethylaniline, Michler's ketone, N-acyl-4-nitro-1-naphthylamine.

Examples of acid-catalyzed cross-linkable groups include, but are not limited to, cyclic ether groups and vinyloxy groups. In an embodiment, the cyclic ether is an epoxide or an oxetane. The photopolymer composition including an acid-catalyzed cross-linkable group typically includes a PAG compound and operates as a “chemically amplified” type of system in a manner described above. Upon exposure to light, the PAG generates an acid (either directly or via a sensitizing dye as described above), which in turn, catalyzes the cross-linking of the acid-catalyzed cross-linkable groups. This significantly changes its solubility relative to the unexposed regions thereby allowing development of an image with the appropriate fluorinated solvent. Usually, cross-linking reduces solubility. In an embodiment, the developing agent includes a fluorinated solvent that selectively dissolves unexposed areas. In an embodiment, a cross-linkable group is provided from a monomer in a weight percentage range of 4 to 40% relative to the copolymer.

Some non-limiting examples of some acid-catalyzed cross-linkable groups include the following wherein (*) refers to an attachment site to the polymer or the polymerizable group of a monomer:

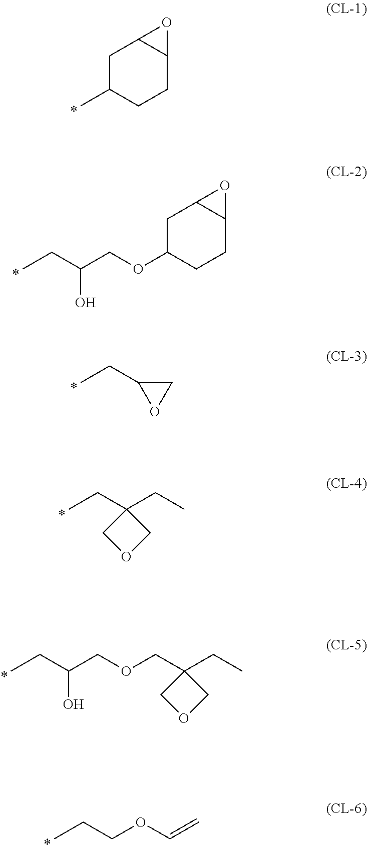

In an embodiment, the solubility-altering reactive groups are ones that, when the photopolymer composition or layer is exposed to light, undergo a bond-breaking reaction to form a material with higher solubility in fluorinated solvents. For example, the solubility-altering reactive groups could be cross-linked and the links are broken upon exposure to light thereby forming lower molecular weight materials. In this embodiment, a fluorinated solvent may be selected to selectively remove exposed areas, thereby acting as a positive photopolymer system.

A combination of multiple second monomers or second repeating units having different solubility-altering reactive groups may be used. For example, a fluorinated photopolymer may comprise both acid-forming and an alcohol-forming precursor groups.

The copolymer may optionally include additional repeating units having other functional groups or purposes. For example, the copolymer may optionally include a repeating unit that adjusts some photopolymer or film property (e.g., solubility, Tg, light absorption, sensitization efficiency, adhesion, surface wetting, etch resistance, dielectric constant, branching and the like).

Many useful PAG compounds exist that may be added to a photopolymer composition. In the presence of proper exposure and sensitization, this photo-acid generator will liberate an acid, which will react with the second monomer portion of the fluorinated photopolymer material to transform it into a less soluble form with respect to fluorinated solvents. The PAG needs to have some solubility in the coating solvent. The amount of PAG required depends upon the particular system, but generally, will be in a range of 0.1 to 6% by weight relative to the copolymer. In an embodiment, the amount of PAG is in a range of 0.1 to 2% relative to the copolymer. Fluorinated PAGs are generally preferred and non-ionic PAGs are particularly useful. Some useful examples of PAG compounds include 2-[2,2,3,3,4,4,5,5-octafluoro-1-(nonafluorobutylsulfonyloxyimino)-pentyl]-fluorene (ONPF) and 2-[2,2,3,3,4,4,4-heptafluoro-1-(nonafluorobutylsulfonyloxyimino)-butyl]-fluorene (HNBF). Other non-ionic PAGS include: norbornene-based non-ionic PAGs such as N-hydroxy-5-norbornene-2,3-dicarboximide perfluorooctanesulfonate, N-hydroxy-5-norbornene-2,3-dicarboximide perfluorobutanesulfonate, and N-hydroxy-5-norbornene-2,3-dicarboximide trifluoromethanesulfonate; and naphthalene-based non-ionic PAGs such as N-hydroxynaphthalimide perfluorooctanesulfonate, N-hydroxynaphthalimide perfluorobutanesulfonate and N-hydroxynaphthalimide trifluoromethanesulfonate. Suitable PAGs are not limited to those specifically mentioned above and some ionic PAGs can work, too. Combinations of two or more PAGs may be used as well.

### REPRESENTATIVE EMBODIMENTS

Below are some non-limiting, representative embodiments of the present disclosure.
1. A method of patterning a bioresorbable material, comprising:
providing a layer of bioresorbable material;
providing a fluoropolymer layer in a first pattern over the layer of bioresorbable material; and
selectively removing the bioresorbable material in areas not covered by the first pattern of fluoropolymer, thereby forming a patterned bioresorbable material.
2. The method according to embodiment 1 wherein the bioresorbable material comprises a polyethylene glycol, a polylactic acid, a polyglycolic acid, a polyvinylalcohol, a polyacrylic acid, a polycaprolactone, a collagen, a polyphosphazene, a polyester-ether, a polyamino acid, or a silk, or a derivative or copolymer thereof.
3. The method according to embodiment 1 or 2, wherein the bioresorbable material further comprises a drug or diagnostic imaging material.
4. The method according to any one of embodiments 1-3 wherein the patterned bioresorbable material has an elongated shape.
5. The method according to embodiment 4 where the patterned bioresorbable material has sufficient mechanical strength to allow insertion into living tissue.
6. The method according to any one of embodiments 1-5 wherein the patterned bioresorbable material has a scaffold shape capable of supporting living tissue or bone growth.
7. The method according to any one of embodiments 1-6 further comprising a bioelectronic device provided under, over, or embedded in, the patterned bioresorbable material.
8. The method according to embodiment 7 wherein the bioelectronic device includes a flexible substructure having a pattern corresponding at least in part to the patterned bioresorbable material.
9. The method according to any one of embodiments 1-8 wherein patterned bioresorbable material is not subsequently exposed to temperature conditions that exceed its Tg.
10. The method according to any one of embodiments 1-9 wherein the fluoropolymer layer is removed after forming the patterned bioresorbable material.
11. The method according to embodiment 10 wherein the patterned fluoropolymer layer is removed by contact with a stripping agent comprising a fluorinated solvent.
12. The method according to any of embodiments 1-11 wherein the layer of bioresorbable material is provided over a carrier support.
13. The method according to embodiment 12 further including removal of the patterned bioresorbable material from the carrier support.
14. The method according to embodiment 13 further comprising providing a release layer between the carrier support and the layer of bioresorbable material, wherein the release layer promotes removal of the patterned bioresorbable material from the carrier support.
15. The method according to any of embodiments 1-14 wherein the bioresorbable material is selectively removed by contact with an etch solvent.
16. The method according to embodiment 15 wherein the etch solvent comprises water, an aliphatic alcohol or chloroform.
17. The method according to any of embodiments 1-14 wherein the bioresorbable material is selectively removed by contact with a plasma etch.
18. The method according to any of embodiments 1-17 wherein the fluoropolymer layer is provided from a composition comprising a fluoropolymer material and a fluorinated solvent.
19. The method according to any of embodiments 1-18, wherein the fluoropolymer layer is a formed from a photosensitive fluoropolymer.
20. The method according to any one of embodiments 1-19 wherein the fluoropolymer layer in a first pattern is provided by applying a composition comprising a photosensitive fluoropolymer material and a fluorinated solvent to form a layer of photosensitive fluoropolymer, exposing the photosensitive fluoropolymer to patterned radiation, and contacting the exposed layer of photosensitive fluoropolymer with a developing agent to form the first pattern of fluoropolymer.
21. The method according to embodiment 20 wherein the photosensitive fluoropolymer material comprises a copolymer having at least two distinct repeating units, including a first repeating unit having a fluorine-containing group and a second repeating unit having a solubility-altering reactive group.
22. The method according to embodiment 21 wherein the solubility-altering reactive group is a carboxylic or sulfonic acid-forming precursor group, an alcohol-forming precursor group or a cross-linking group.
23. The method according to any of embodiments 19-21 wherein the developing agent includes at least 50% by volume of a fluorinated solvent.
24. The method according to embodiment 23, wherein the fluorinated solvent includes a hydrofluoroether.
25. The method according to any of embodiments 19-24 wherein the layer of photosensitive fluoropolymer layer includes a non-ionic, fluorinated photo-acid generator compound.
26. The method according to any of embodiments 1-24 wherein the total fluorine content of the photosensitive fluoropolymer is in a weight range of 15 to 60%.
27. A method of forming a patterned bioelectronic device, comprising:
providing a flexible substructure having at least one bioelectronic feature, at least a portion of which is electrically conductive or semiconductive;
providing a layer of bioresorbable material over the flexible substructure including the bioelectronic feature;
providing a layer of a fluoropolymer in a first pattern covering the bioresorbable material thereby forming a second pattern of non-covered bioresorbable material; and
selectively removing the second pattern of non-covered bioresorbable material, thereby forming a layer of patterned bioresorbable material corresponding to the first pattern and a pattern of uncovered substructure corresponding to the second pattern.
28. The method of embodiment 27 further comprising selectively removing at least a portion of the uncovered substructure, thereby forming a patterned bioelectronic device having a patterned substructure corresponding to the first pattern.
29. The method according to embodiment 27 wherein the bioresorbable material comprises a polyethylene glycol, a polylactic acid, a polyglycolic acid, a polyvinylalcohol, a polyacrylic acid, a polycaprolactone, a collagen, a polyphosphazene, a polyester-ether, a polyamino acid, or a silk, or a derivative or copolymer thereof.
30. The method according to any of embodiments 27-28 wherein the bioresorbable material further comprises a drug or diagnostic imaging material.
31. The method according to any one of embodiments 27-30 wherein the total fluorine content of the fluoropolymer is in a weight range of 15 to 60%.
32. The method according to any one of embodiments 27-31 wherein the non-covered bioresorbable material is selectively removed by contact with an etch solvent.
33. The method according to any of embodiments 27-30 wherein the non-covered bioresorbable material is selectively removed by contact with a plasma etch.
34. The method according to any of embodiments 28-33 wherein the uncovered substrate is selectively removed by contact with a plasma etch.
35. The method according to any one of embodiments 27-34 wherein the patterned bioresorbable material is not subsequently exposed to temperature conditions that exceed its Tg.
36. The method according to any one of embodiments 28-35 wherein the first pattern of fluoropolymer is removed after forming the patterned substrate.
37. The method according to embodiment 36 wherein the first pattern of fluoropolymer is removed by contact with a stripping agent comprising a fluorinated solvent.
38. The method according to any of embodiments 27-37 wherein the flexible substructure is provided over a carrier support.
39. The method according to embodiment 38 further comprising providing a release layer between the carrier support and the flexible substructure.
40. The method according to any one of embodiments 27-39 wherein the bioelectronic feature includes a conductive polymer, a metal electrode, a light-emitting material, an organic semiconductor or combinations thereof.
41. The method according to any one of embodiments 27-40 wherein the bioelectronic feature forms at least a portion of a biosensor, a bio-stimulator, or an ion pump.
42. The method according to any one of embodiments 27-41 wherein the bioelectronic feature forms at least a portion of an organic electrochemical transistor.
43. The method according to any one of embodiments 27-42 wherein the substrate comprises a parylene, a second fluoropolymer, or combinations thereof
44. The method according to any one of embodiments 27-43 wherein the layer of fluoropolymer in a first pattern is provided by applying a composition comprising a photosensitive fluoropolymer material and a fluorinated solvent to form a layer of photosensitive fluoropolymer, exposing the photosensitive fluoropolymer to patterned radiation, and contacting the exposed layer of photosensitive fluoropolymer with a developing agent to form the first pattern of fluoropolymer.
45. The method according to embodiment 44 wherein the photosensitive fluoropolymer material comprises a copolymer having at least two distinct repeating units, including a first repeating unit having a fluorine-containing group and a second repeating unit having a solubility-altering reactive group.
46. The method according to embodiment 45 wherein the solubility-altering reactive group is a carboxylic or sulfonic acid-forming precursor group, an alcohol-forming precursor group or a cross-linking group.
47. The method according to any of embodiments 44-46 wherein the developing agent includes at least 50% by volume of a fluorinated solvent.
48. The method according to embodiment 47, wherein the fluorinated solvent includes a hydrofluoroether.
49. The method according to any of embodiments 44-48 wherein areas of the photosensitive fluoropolymer receiving the patterned radiation are transformed into a structure having a lower solubility in the developing agent than areas that did not receive the radiation.
50. The method according to any of embodiments 27-43 wherein the layer of fluoropolymer in a first pattern is provided by:
a) applying a layer of non-patterned fluoropolymer over the bioresorbable material, wherein the fluoropolymer is soluble in a fluorinated solvent;
b) providing over the non-patterned fluorinated polymer a layer of a second polymer in a pattern corresponding to the first pattern to form a partially patterned bilayer polymer structure, wherein the second polymer is substantially insoluble in the fluorinated solvent; and
c) contacting the partially patterned bilayer polymer structure with the fluorinated solvent to selectively remove the fluoropolymer in areas other than the first pattern.
51. The method according to embodiment 50 wherein the second polymer is a photoresist having a total fluorine content by weight of less than 30% and the fluoropolymer has a total fluorine content by weight of greater than 40%.
52. A bioelectronic device comprising:
a flexible substructure including a bioactive portion having a first side and a second side, wherein the bioactive portion is provided in a first pattern and includes at least one bioelectronic feature, at least a portion of which is conductive or semiconductive, provided as part of the first side; and
a layer of bioresorbable material provided over the first side of the bioactive portion in a pattern corresponding to the first pattern,
wherein the bioelectronic device includes substantially no bioresorbable material on the second side of the bioactive portion.
53. The bioelectronic device of embodiment 52 wherein the patterned layer of bioresorbable material is in contact with the bioelectronic feature.
54. The bioelectronic device of embodiment 53 wherein the patterned layer of bioresorbable material includes an opening in alignment with the bioelectronic feature so that the bioresorbable material is not in full contact with the bioelectronic feature.
55. The bioelectronic device of any of embodiments 52-54, wherein the flexible substructure further includes an electrical contact portion in electrical communication with the bioactive portion.
56. The bioelectronic device of embodiment 55 wherein the first pattern of bioactive portion includes an elongated shape and the bioelectronic feature is provided at a distal end of the elongated shape relative to the electrical contact portion.
57. A method of forming a patterned bioelectronic device, comprising:
providing a layer of bioresorbable material;
forming a flexible substructure in a first pattern over the layer of bioresorbable material, the flexible substructure comprising at least one patterned fluoropolymer layer and at least one bioelectronic feature, at least a portion of which is electrically conductive or semiconductive; and
selectively removing the bioresorbable material in areas not covered by the first pattern, thereby forming a patterned bioelectronic device having bioresorbable material in a pattern corresponding to the first pattern.
58. The method according to embodiment 57 wherein the bioresorbable material comprises a polyethylene glycol, a polylactic acid, a polyglycolic acid, a polyvinylalcohol, a polyacrylic acid, a polycaprolactone, a collagen, a polyphosphazene, a polyester-ether, a polyamino acid, or a silk, or a derivative or copolymer thereof
59. The method according to embodiment 58, wherein the bioresorbable material further comprises a drug or diagnostic imaging material.
60. The method according to any one of embodiments 57-59 wherein the total fluorine content of the fluoropolymer is in a weight range of 15 to 60%.
61. The method according to any one of embodiments 57-60 wherein non-covered bioresorbable material is selectively removed by contact with an etch solvent.
62. The method according to embodiment 61 wherein the etch solvent comprises water, an aliphatic alcohol or chloroform.
63. The method according to any of embodiments 57-60 wherein non-covered bioresorbable material is selectively removed by contact with a plasma etch.
64. The method according to any one of embodiments 57-63 wherein the patterned bioresorbable material is not exposed to temperature conditions that exceed its Tg.
65. The method according to any of embodiments 57-64 wherein the layer of bioresorbable material is provided over a carrier support.
66. The method according to embodiment 65 further including removal of the patterned bioelectronic device from the carrier support.
67. The method according to embodiment 66 further comprising providing a release layer between the carrier support and the bioresorbable material, wherein the release layer promotes removal of the patterned bioelectronic device from the carrier support.
68. The method according to any one of embodiments 57-67 wherein the bioelectronic feature includes a conductive polymer, a metal electrode, a light-emitting material, an organic semiconductor or combinations thereof.
69. The method according to any one of embodiments 57-68 wherein the bioelectronic feature forms at least a portion of a biosensor, a bio-stimulator, an organic electrochemical transistor, or an ion pump.
70. The method according to any one of embodiments 57-69 wherein the patterned fluoropolymer defines the first pattern.
71. The method according to embodiment 70 wherein the patterned fluoropolymer is formed from multiple layers of one or more fluoropolymer materials that have the same or different composition.
72. The method according to any one of embodiments 57-71 wherein the patterned fluoropolymer is provided at least in part from one or more photosensitive fluoropolymers.
73. The method according to embodiment 72 wherein providing the patterned fluoropolymer includes applying a composition comprising a photosensitive fluoropolymer material and a fluorinated solvent to form a layer of photosensitive fluoropolymer, exposing the photosensitive fluoropolymer to patterned radiation to form an exposed layer of photosensitive fluoropolymer, and contacting the exposed layer of photosensitive fluoropolymer with a developing agent to form the patterned fluoropolymer.
74. The method according to embodiment 73 wherein the photosensitive fluoropolymer material comprises a copolymer having at least two distinct repeating units, including a first repeating unit having a fluorine-containing group and a second repeating unit having a solubility-altering reactive group.
75. The method according to embodiment 74 wherein the solubility-altering reactive group is a cross-linking group.
76. The method according to any of embodiments 73-75 wherein the developing agent includes at least 50% by volume of a fluorinated solvent.
77. The method according to embodiment 76, wherein the fluorinated solvent includes a hydrofluoroether.
78. The method according to any of embodiments 57-71 wherein at least a portion of the patterned fluoropolymer is provided by:
a) applying a layer of non-patterned fluoropolymer over the bioresorbable material, wherein the fluoropolymer is soluble in a fluorinated solvent;
b) providing over the non-patterned fluorinated polymer a layer of a second polymer in a predetermined pattern to form a partially patterned bilayer polymer structure, wherein the second polymer is substantially insoluble in the fluorinated solvent; and
c) contacting the partially patterned bilayer polymer structure with the fluorinated solvent to selectively remove the fluoropolymer in areas other than the predetermined pattern.
79. The method according to embodiment 79 wherein the second polymer is a photoresist having a total fluorine content by weight of less than 30% and the fluoropolymer has a total fluorine content by weight of greater than 40%.
80. A bioelectronic device comprising:
a flexible substructure including a bioactive portion having a first side and a second side, wherein the bioactive portion is provided in a first pattern and includes at least one bioelectronic feature, at least a portion of which is conductive or semiconductive, provided as part of the first side; and
a layer of bioresorbable material provided on the second side of the bioactive portion in a pattern corresponding to the first pattern,
wherein the bioelectronic device includes substantially no bioresorbable material on the first side of the bioactive portion.
81. The bioelectronic device of embodiments 80, wherein the flexible substructure further includes a electrical contact portion in electrical communication with the bioactive portion.
82. The bioelectronic device of embodiment 81 wherein the first pattern of bioactive portion includes an elongated shape and the bioelectronic feature is provided at a distal end of the elongated shape relative to the electrical contact portion.

### EXAMPLES

### Example 1

An approximately 1 μm film of polyethylene glycol (PEG) was prepared on a UV/ozone-cleaned glass slide by spin coating a 10% w/w aqueous solution of PEG (Aldrich, mw=20,000) and drying on a hot plate for 1 min at 90° C. Over the PEG film, OSCoR 3313 photoresist (from Orthogonal, Inc.) was coated at 1000 rpm and soft baked at 90° C. for 20 seconds. OSCoR 3313 is a photosensitive fluorinated photopolymer provided in a hydrofluoroether solvent along with a fluorinated non-ionic PAG. The fluorine content of the photosensitive fluoropolymer was about 41% by weight and the polymer included both a carboxylic acid-forming precursor group and an alcohol-forming precursor group. The film thickness was about 1.3 μm.

The slide was exposed to patterned i-line radiation with a total dose of about 72 mJ/cm² and given a post exposure bake of 90° C. for 20 sec. This was followed by development using two (2) 1 min puddles and one (1) 30 sec puddle of Orthogonal Developer 100 (comprises a hydrofluoroether solvent that is different from the OSCoR 3313 coating solvent), each followed by spin dry step, to form a patterned OSCoR 3313 layer having a plurality of rectangular openings about 0.8 by 1.5 mm in dimension.

Next, PEG in areas not covered by the patterned OSCoR 3313 were wet etched (removed) by contact with water for about 10 sec to form a pattern of PEG corresponding to the pattern of OSCoR 3313 (the OSCoR 3313 is not soluble in water). The OSCoR 3313 was removed using Orthogonal Stripper 700 (1 min) followed by a spin dry step.

The above example shows that the patterned fluoropolymer makes an effective resist for patterning a bioresorbable material. Although the contact angle for water on OSCoR 3313 is very high (i.e., it is very hydrophobic), the fluoropolymer surprisingly coats very well onto the hydrophilic polymer from the fluorinated solvent coating solution, in this case a hydrofluoroether. Further, adhesion is surprisingly sufficient to permit wet etch patterning in water.

### Comparison 1

A PEG film was treated with MF26A developer, a common developing agent for conventional positive tone resists such as Shipley S1813. The PEG film was immediately attacked and dissolved by this developer, which shows that such a system would not be suitable for photolithographic patterning of the bioresorbable material.

### Example 2

An aqueous solution was prepared containing 14% by weight polyvinyl alcohol (“PVOH”, 88% hydrolyzed, MW=13K-17K) and 2% by weight n-butanol as a coating aid. A silicon wafer chip approximately 2 cm×2 cm was cleaned with an ammonia-based detergent, rinsed with deionized water and dried. A film of PVOH was prepared by spin-coating the PVOH solution onto the silicon chip at 1000 rpm for 1 minute followed by a 5 minute post-apply bake (PAB) on a 90° C. hot plate. The film thickness was determined to be about 1.8 μm as measured on a Filmetrics F20 Thin-Film Analyzer. A layer of about 1.5 um OSCoR 4000 photoresist (from Orthogonal, Inc.) was spin-coated over the PVOH layer at 1000 rpm for 1 minute, followed by a 1 minute bake on a 90° C. hot plate. OSCoR 4000 is a photosensitive fluorinated photopolymer provided in a hydrofluoroether solvent along with a fluorinated non-ionic PAG. The fluorine content of the photosensitive fluoropolymer was about 42% by weight and the polymer included a carboxylic acid-forming precursor group. OSCoR 4000 coats surprisingly well over the PVOH, but does not dissolve or otherwise damage the PVOH.

Next, half of the silicon chip was covered with an opaque mask and the bilayer structure was exposed to UV radiation using a Pluvex 1410 UV exposure unit having a maximum emission intensity at 365 nm. The dose was about 120 mJ/cm² as measured at 365 nm. Following exposure, the silicon chip was heated for 1 minute on a 90° C. hot plate and then developed by applying three consecutive “puddles” of Developer 103 from Orthogonal, Inc. Developer 103 includes a hydrofluoroether developing agent. Each puddle had a dwell time of 30 sec and the sample was spin-dried between puddle applications. Development removed the unexposed half of the OSCoR 4000 thereby revealing the underlying PVOH layer in this region. The exposed portion remained over the PVOH.

The sample was then immersed in deionized water for 15 sec and blown dry. Most of the PVOH was removed in the unexposed areas after this first immersion. The process was repeated three (3) more times to ensure full removal of PVOH, thereby forming a bare silicon surface in the unexposed area. The OSCoR 4000 was stripped using Stripper 700 from Orthogonal, Inc. (also comprising a hydrofluoroether solvent) by applying three puddles for 30 sec each in the same way described above for the development. This forms a patterned bioresorbable PVOH polymer in a pattern corresponding to the area of exposure.

The above example again shows that the patterned fluoropolymer makes an effective resist for patterning a bioresorbable material. Although the contact angle for water on OSCoR 4000 is very high (i.e., it is very hydrophobic), the fluoropolymer surprisingly coats very well onto the hydrophilic polymer from the fluorinated solvent coating solution, in this case a hydrofluoroether. Further, adhesion is surprisingly sufficient to permit wet etch patterning in water.

### Comparison 2

A 1.8 μm PVOH film was prepared as above and coated with nLOF 2020 (AZ Electronic Materials) to a thickness of about 2.3 μm. Next, half of the silicon chip was covered with an opaque mask and the bilayer structure was exposed to UV radiation using a Pluvex 1410 UV exposure unit having a maximum emission intensity at 365 nm. The dose was about 160 mJ/cm² as measured at 365 nm. Following exposure, the silicon chip was heated for 1 minute on a 110° C. hot plate and then developed by applying three consecutive “puddles” of AZ MIF 300 developer. Each puddle had a dwell time of 30 sec and the sample was spin-dried between puddle applications. Development removed the unexposed half of the nLOF 2020, but also attacked the underlying PVOH and started undercutting the PVOH under the exposed nLOF. Although the intention was to remove the PVOH in the unexposed areas, the MIF 300 developer is very aggressive (more aggressive than DI water) and it is difficult to control both the photoresist development profile and the PVOH patterning in a single step. With the patterned fluoropolymer example, the developing agent is orthogonal to the PVOH, and PVOH etching can be carefully controlled in a separate step.

The invention has been described in detail with particular reference to certain embodiments thereof, but it will be understood that variations, combinations, and modifications can be effected by a person of ordinary skill in the art within the spirit and scope of the invention.

### LIST OF REFERENCE NUMBERS USED IN THE DRAWINGS

2 provide layer of bioresorbable material step
4 provide fluoropolymer layer in a first pattern step
6 selectively remove bioresorbable material step
20 layer of bioresorbable material
22 carrier substrate
24 release layer
28 patterned bioresorbable material
60 photosensitive fluoropolymer
61 radiation
62 photomask
63 exposed layer of photosensitive fluoropolymer
64 pattern of exposed photosensitive fluoropolymer
65 pattern of unexposed photosensitive fluoropolymer
66 first pattern of fluoropolymer
101 provide flexible substructure having at least one bioelectronic feature step
103 provide layer of bioresorbable material step
105 provide first pattern of fluoropolymer step
107 selectively remove non-covered bioresorbable material step
109 selectively remove at least a portion of the uncovered substrate step
110 flexible support
111 protected flexible substructure
112 flexible substructure
113 uncovered substructure
114 patterned flexible support
115 patterned substructure
115-1 first side of patterned substructure
115-2 second side of patterned substructure
116 patterned, protected flexible substructure
123 metal electrode
130 patterned electrical insulator
132 photopolymer
140 conductive polymer
160 non-patterned fluoropolymer
161 photosensitive second polymer
163 exposed layer of photosensitive second polymer
164 pattern of exposed photosensitive second polymer
165 pattern of unexposed photosensitive second polymer
166 patterned layer of second polymer
170 protection layer
220 layer of bioresorbable material
221 second pattern of non-covered bioresorbable material
228 patterned layer of bioresorbable material
266 first pattern of fluoropolymer
270 patterned bioelectronic device
271 patterned bioelectronic device
272 patterned bioelectronic device
273 patterned bioelectronic device
301 first intermediate structure
302 second intermediate structure
303 third intermediate structure
304 array of patterned bioelectronic devices
315 patterned flexible substructure
315-1 first side of patterned flexible substructure
315-2 second side of patterned flexible substructure
328 patterned bioresorbable material
366 first pattern of fluoropolymer
381 electrical contact pads
382 conductive traces
383 electrode pad
384 patterned insulating layer
385 modifying material
391 patterned bioelectronic device
395 electrical contact portion
398 shank portion
402 provide layer of bioresorbable material step
404 provide flexible substructure in first pattern step
406 selectively remove bioresorbable material step
415 patterned flexible substructure
415-1 first side of patterned flexible substructure
415-2 second side of patterned flexible substructure
416 protected flexible substructure
420 layer of bioresorbable material
423 metal electrode
428 patterned bioresorbable material
430 patterned electrical insulator layer
440 conductive polymer
466 patterned fluoropolymer layer
470 protection layer
472 patterned bioelectronic device
473 patterned bioelectronic device

1. A method of patterning a bioresorbable material, comprising:
providing a layer of bioresorbable material;
providing a fluoropolymer layer in a first pattern over the layer of bioresorbable material; and
selectively removing the bioresorbable material in areas not covered by the first pattern of fluoropolymer, thereby forming a patterned bioresorbable material.

2. The method according to claim 1 wherein the bioresorbable material comprises a polyethylene glycol, a polylactic acid, a polyglycolic acid, a polyvinyl alcohol, a polyacrylic acid, a polycaprolactone, a collagen, a polyphosphazene, a polyester-ether, a polyamino acid, or a silk, or a derivative or copolymer thereof.

3. The method according to claim 1 further comprising a bioelectronic device provided under, over, or embedded in, the patterned bioresorbable material.

4. The method according to claim 3 wherein the bioelectronic device includes a flexible substructure having a pattern corresponding at least in part to the patterned bioresorbable material.

5. The method according to claim 1 wherein the fluoropolymer layer is removed after forming the patterned bioresorbable material by contact with a stripping agent comprising a fluorinated solvent.

6. The method according to claim 1 wherein the layer of bioresorbable material is provided over a carrier support and the method further includes removal of the patterned bioresorbable material from the carrier support.

7. The method according to claim 1 wherein the bioresorbable material is selectively removed by contact with an aqueous or polar organic etch solvent.

8. The method according to claim 1 wherein the bioresorbable material is selectively removed by contact with a plasma etch.

9. The method according to claim 1, wherein the fluoropolymer layer in a first pattern is a formed from a photosensitive fluoropolymer.

10. The method according to claim 1 wherein the total fluorine content of the fluoropolymer is in a weight range of 15 to 60%.

11. A method of forming a patterned bioelectronic device, comprising:
providing a flexible substructure having at least one bioelectronic feature, at least a portion of which is electrically conductive or semiconductive;
providing a layer of bioresorbable material over the flexible substructure including the bioelectronic feature;
providing a layer of a fluoropolymer in a first pattern covering the bioresorbable material thereby forming a second pattern of non-covered bioresorbable material, wherein the total fluorine content of the fluoropolymer is in a weight range of 15 to 60%; and
selectively removing the second pattern of non-covered bioresorbable material, thereby forming a layer of patterned bioresorbable material corresponding to the first pattern and a pattern of uncovered substructure corresponding to the second pattern.

12. The method of claim 11 further comprising selectively removing at least a portion of the uncovered substructure, thereby forming a patterned bioelectronic device having a patterned substructure corresponding to the first pattern.

13. The method claim 12 wherein the uncovered substrate is selectively removed by contact with a plasma etch.

14. The method according to claim 11 wherein the bioresorbable material comprises a polyethylene glycol, a polylactic acid, a polyglycolic acid, a polyvinyl alcohol, a polyacrylic acid, a polycaprolactone, a collagen, a polyphosphazene, a polyester-ether, a polyamino acid, or a silk, or a derivative or copolymer thereof.

15. The method according to claim 11 wherein the non-covered bioresorbable material is selectively removed by contact with an aqueous or polar organic etch solvent or by contact with a plasma etch.

16. The method according to claim 1 wherein the bioelectronic feature includes a conductive polymer, a metal electrode, a light-emitting material, an organic semiconductor or combinations thereof, and wherein the bioelectronic feature forms at least a portion of a biosensor, a bio-stimulator, an organic electrochemical transistor or an ion pump.

17. The method according to claim 1 wherein the layer of fluoropolymer in a first pattern is provided by applying a composition comprising a photosensitive fluoropolymer material and a fluorinated solvent to form a layer of photosensitive fluoropolymer, exposing the photosensitive fluoropolymer to patterned radiation, and contacting the exposed layer of photosensitive fluoropolymer with a developing agent comprising a fluorinated solvent to form the first pattern of fluoropolymer.

18. A bioelectronic device comprising:
a flexible substructure including a bioactive portion having a first side and a second side, wherein the bioactive portion is provided in a first pattern and includes at least one bioelectronic feature, at least a portion of which is conductive or semiconductive, provided as part of the first side; and
a layer of bioresorbable material provided over the first side of the bioactive portion in a pattern corresponding to the first pattern,
wherein the bioelectronic device includes substantially no bioresorbable material on the second side of the bioactive portion.

19. The bioelectronic device of claim 18 wherein the patterned layer of bioresorbable material includes an opening in alignment with the bioelectronic feature so that the bioresorbable material is not in full contact with the bioelectronic feature.

20. The bioelectronic device of claim 18, wherein the flexible substructure further includes an electrical contact portion in electrical communication with the bioactive portion, and wherein the first pattern of bioactive portion includes an elongated shape and the bioelectronic feature is provided at a distal end of the elongated shape relative to the electrical contact portion.
